(19) United States
(12) Patent Application Publication
(10) Pub. No.: US 2026/0183420 A1
(43) Pub. Date: Jul. 2, 2026

(54) ACTIVATED NANOTHERAPY FOR SICKLE CELL DISEASE

(71) Applicants: UNIVERSITY OF MARYLAND, BALTIMORE, Baltimore, MD (US); UNIVERSITY OF MARYLAND, BALTIMORE COUNTY, Baltimore, MD (US)

(72) Inventors: Dipanjan PAN, State College, PA (US); Allan DOCTOR, Towson, MD (US); Parikshit MOITRA, State College, PA (US)

(21) Appl. No.: 19/131,255

(22) PCT Filed: Nov. 22, 2023
(86) PCT No.: PCT/US23/81009

§ 371 Date: May 20, 2025

Related U.S. Application Data
(60) Provisional application No. 63/384,642, filed on Nov. 22, 2022.

(51) Int. Cl. A61K 47/69; A61K 47/54; A61P 7/06; B82Y 5/00

(52) U.S. Cl. CPC A61K 47/6929; A61K 47/544; A61P 7/06; B82Y 5/00

(57) ABSTRACT

We disclose here the nano-enabled delivery of highly potent 5-hydroxymethyl furfural (5-HMF) to sickled blood cells in patients. 5-HMF, a superior antisickling agent, has been formulated to address the limitations of existing treatment modalities arising from disfavorable pharmacokinetics of the drug compound. Specifically, two complementary 5-HMF prodrugs are integrated as a ‘protected’ biocompatible composite nanoparticle designed to readily fuse with RBCs and be amenable to transdermal delivery (5-HMF-grafted-phospholipid layered over a sucrose-derived graphitic carbon dot core). These RBC-targeted, protected, biocompatible, self-assembled 5-HMF prodrug nanoparticles for sickle cell disease are the first in class technology for offering a treatment for sickle cell disease which has improved potency, targeted payload delivery, and extended release with the red blood cells with enhanced pharmacodynamic effect in a treated patient.

### CROSS-REFERENCE TO RELATED APPLICATIONS

This application is a 371 National Stage Application of PCT Application PCT/US23/81009 filed 22 Nov. 2023, which claims the benefit of U.S. Provisional Application Ser. No. 63/384,642, filed 22 Nov. 2022. The entire contents of each of these applications is hereby incorporated by reference as if fully set forth herein.

### BACKGROUND OF THE INVENTION

### 1. Field of the Invention

The invention pertains to the field of medicine and medical treatment. In particular, the invention relates to a nanotherapy for sickle cell disease.

### 2. Background of the Invention

Sickle cell disease (SCD) is a group of genetic blood disorders in which the hemoglobin is damaged and cannot carry oxygen to the tissues. Sickle cell anemia results in an abnormality in the oxygen-carrying hemoglobin found in red blood cells. This leads to a rigid, sickle-like shape under certain circumstances.

Sickle cell disease pathology stems from deoxygenation-induced hemoglobin (HbS) polymerization resulting from the substitution of glutamic acid for valine at the sixth amino acid of β-globin protein. Following deoxygenation in red blood cells (RBCs), HbS forms polymers, causing the RBCs to become deformed and adherent, leading to vaso-occlusive events and hemolysis that result in splenic infarction, kidney failure, stroke, painful crises, chronic anemia, and decreased survival.

It is estimated that the disease affects approximately 100,000 Americans, occurs in about 1 out of every 365 Black or African-American people, occurs in about 1 out of every 16,300 Hispanic-American people. About 1 in 13 Black or African-American babies is born with the genetic sickle cell trait (SCT).

Normal hemoglobin (Hb) contains two alpha and two beta chains (α₂β₂), while hemoglobin with the sickle cell trait (heterozygous) contains two alpha, one beta, and one abnormal chain (α₂βS), and hemoglobin homozygous for the sickle cell trait contains two alpha and two abnormal chains (α₂SS). Sickle cell disease occurs when a person inherits two abnormal copies of the beta-globin gene, one from each parent. A person with a single abnormal copy usually does not have symptoms and is said to carry the sickle cell trait.

There are only four FDA approved drugs for SCD; only one (Voxelotor) directly targets HbS polymerization. Voxelotor (among the aromatic aldehyde class of Hb O2 affinity modifying agents) advanced primarily due to tractable pharmacokinetics (oral, daily dosing) rather than by superior anti-sickling potency. Currently, the cytotoxic drug, hydroxyurea (hydroxycarbamide), is the only FDA-approved drug that is being used clinically for sickle cell disease. In some patients, this drug can reduce the frequency of painful episodes and the risk of life-threatening illness but important side effects, for example bone marrow effects, can reduce its uses. Hydroxyurea is not effective in a large fraction of patients and therefore new agents are currently needed. In summary, hydroxyurea therapy is associated with various undesirable side effects, and not all patients benefit from this treatment. Despite considerable effort, however, there has been little progress in the development of new anti-sickling agents that have efficacy and safety in vivo.

Allosteric Hb adducts that increase oxyHbS abundance target a mechanistic trigger of the disease. The drug 5-hydroxymethylfurfural (5-HMF) binds to Hb NH₂ terminal αValine1, resulting in increases in O₂ affinity, reductions in sickling and protecting hypoxic Townes sickle cell (SS) mice (survival, organ injury). However, 5-HMF did not advance beyond Phase I/II clinical trials due to pharmacokinetic/dynamic (PK/PD) limitations. A related molecule (Voxelotor) was approved based on surrogate endpoints (Hb level and hemolytic markers), but without decreasing vaso-occlusive morbidities. These therapies have therefore been inadequate to treat the condition. Currently, there are no known nanotherapeutics for SCDs.

### SUMMARY OF THE INVENTION

Thus, there exists a need in the art for new therapeutic modalities for sickle cell disease. In recognition of the critical need for new SCD treatments, in 2019 the FDA granted Voxelotor (Oxbryta) Breakthrough Therapy, Fast Track, Orphan Drug, and Rare Pediatric Disease designations for the treatment of patients (12 y and older) with SCD. In 2021, the FDA approved a supplemental NDA for expanded indication of Voxelotor (Oxbryta) for children as young as 4 years of age with SCD. Of note, the FDA's regulations allow approval of a drug based only on surrogate endpoints (here improving anemia and reducing background hemolysis) if the drug addresses an unmet medical need and the condition is serious. It is important to recognize that long term follow up (72 weeks, HOPE trial) did not demonstrate modification of typical SCD morbidities (organ injury); moreover, there are concerns with patient compliance (for Voxelotor) arising from the incidence of (primarily) diarrhea and headache. As such, as described in our project plan, alternative antisickling drugs (e.g. reformulated 5-HMF) would outperform Voxelotor based upon (a) improved efficacy and/or (b) improved compliance (from

The invention described herein relates to compositions and methods for treatment of sickle cell disease. In particular embodiments, the present invention relates to a nanoparticle, comprising: (a) a carbon core comprising a carbon dot and complexed 5-HMF; and (b) a 5-HMF-phospholipid shell comprising a lipid complexed to 5-HMF, wherein the lipid complex is cleavable by PLA₂. In particular embodiments, the nanoparticle is about 0.1 nm to about 200 nm in diameter, or about 1 nm to about 100 nm in diameter, or about 50 nm to about 100 nm in diameter.

In certain embodiments of the invention, the phospholipid is an oxidized lipid, and preferably is 1-lysophosphatidylcholine.

In certain embodiments of the invention relates to a nanoparticle pharmaceutical composition comprising a pharmaceutically acceptable vehicle and the nanoparticle described above. In some embodiments, composition is formulated for transdermal administration.

In certain embodiments of the invention relates to a method of treating sickle cell disease in a subject in need thereof, comprising administering to the subject the nanoparticle or the nanoparticle pharmaceutical composition described above.

### BRIEF SUMMARY OF THE DRAWINGS

Certain embodiments are illustrated by way of example, and not by way of limitation, in the figures of the accompanying drawings.

FIG. 1 is a schematic diagram showing example carbon dot nanoparticles (CNP).

FIG. 2 is a diagram showing the design and production of a prodrug composition that enables delivery of anti-sickling agents, e.g., 5-hydroxymethyl furfural (5-HMF).

FIG. 3 is a drawing showing the synthesis of 5-HMF phospholipid prodrugs.

FIG. 4 is a drawing showing an alternative synthetic route to produce 5-HMF-prodrugs.

FIG. 5 is an example of a graph showing parameters of osmotic fragility.

FIG. 6 is a graph showing a sample RBC deformability curve and the calculation of elongation index.

FIG. 7 is a graph showing an example calculation of Oxyscan™ analysis parameters.

FIG. 8 is a graph showing generic calculations for refining the point of sickling (POS).

FIG. 9 is a graph showing an example POS determination from first derivative.

FIG. 10 is a western blot showing the measurement of PLA2 activity in erythrocytes.

FIG. 11 shows a chemical synthetic scheme for 5-HMF prodrug.

FIG. 12 presents information on characterization of 5-HMF-prodrug and 5-HMF composite nanoparticles (CompNp). FIG. 12A shows overlaid ¹H-NMR spectra of 5-HMF-prodrug and lipid precursor to show successful synthesis (¹H NMR (400 MHz, CDCl₃) spectra (Red) of the compound and LysoPC (black)); FIG. 12B is a MALDI MS spectrum of the 5-HMF-prodrug; FIG. 12C and FIG. 12D present anhydrous state transmission electron microscopy (TEM) images of lipid-coated CDs and 5-HMF-prodrug-lipid coated CDs (carbon grid).

FIG. 13 is a diagram showing the lipase catalyzed biotransformation of carbon dots.

FIG. 14A and FIG. 14B are graphs showing the absorbance characteristics of four different nanoparticles (absorbance and intensity, respectively). FIG. 14C shows the physical appearance of the nanoparticles.

FIG. 15A and FIG. 15B are bar graphs showing the average hydrodynamic diameter and Zeta potential of the indicated nanoparticles.

FIG. 16A shows the Raman spectra of the CD-PEG samples in their pristine condition, subjected to lipase-peroxide oxidation for 24 hours and one week respectively. FIG. 16B shows the FTIR absorbance spectra of the particles.

FIG. 17A, FIG. 17B, FIG. 17C, and FIG. 17D show the effect of enzymatic degradation on morphological characteristics of CDs. FIG. 17A and FIG. 17B are TEM photographs of pre-enzymatically treated samples. FIG. 17C and FIG. 17D are TEM photographs of post-enzymatically treated samples. FIG. 17E is a graph showing a comparative analysis between circularity parameter and maximum height of nanoparticles (nm) obtained from AFM studies.

FIG. 18A shows the degradation of the nanoparticle monitored by MALDI/TOF spectrometry at different time points. FIG. 18B shows the degradation of nanoparticles monitored by ¹H NMR spectroscopy. FIG. 18C shows the sequential oxidation process of sucrose and glucose leading to the formation of different intermediate products. FIG. 18D shows the degradation of sucrose in combined presence of Lipase and H₂O₂, leading to the formation of intermediate products.

FIG. 19A is a graph showing the modified Hb % after the indicated dose of 5-HMF incubated in vitro for one hour. FIG. 19B presents illustrative chromatograms (HPLC tracings—standards & samples). FIG. 19C presents data concerning % 5-HMF modified Hb. FIG. 19D shows data concerning HbO₂ affinity (p50, O2 partial pressure at which Hb is 50% saturated). The effect of 5-HMF upon hypoxia-induced sickling was determined by laser ektacytometry during controlled SRBC deoxygenation (LORRCA Oxygenscan), which identifies the pO₂ at which RBC deformability maximally changes upon (FIG. 19E) deoxygenation (point of sickling, POS) and (FIG. 19F) reoxygenation (point of recovery (POR)).

FIG. 20A through FIG. 20K are a set of graphs showing incubation time response. FIG. 20A (p50); FIG. 20B (Hill number (mean)); FIG. 20C (HEMOX traces time); FIG. 20D (POS 1^{st} derivative>4×10⁻³); FIG. 20E (EImax); FIG. 20F (EImin); FIG. 20G (delta EI): FIG. 20H (area); FIG. 20I (area between curves); FIG. 20J (POR 1^{st} derivative >0.235×10³); and FIG. 20K (recovery).

FIG. 21A and FIG. 21B (control patients) and FIG. 21C and FIG. 21D (SCD patients) present data on human RBCs.

FIG. 22 presents data on human SCD RBCs were incubated with 5-HMF or 5-HMF prodrug formulations. FIG. 22A: O2 unloading; FIG. 22B: improved hypoxia tolerance; FIG. 22C: p50 during O2 loading; FIG. 22D): reoxygenation induced point of recovery at lower pO₂.

FIG. 23A and FIG. 23B present data on O₂ loading.

FIG. 24 is a table showing patient supplement intake.

FIG. 25 is a table showing patient analgesic use.

FIG. 26 is a table showing patient medications (A-C).

FIG. 27 is a table showing patient medications (D-I).

FIG. 28 is a table showing patient medications (J-N).

FIG. 29 is a table showing patient medications (P-Z).

FIG. 30A, FIG. 30B, and FIG. 30C are graphs showing the point of sickling versus the dose of 5-HMF, as shown.

FIG. 31 is a graph showing an example of refining the point of sickling (LORRCA).

FIG. 32 is a graph showing the point of recovery (POR) determined from 1^{st} derivative.

FIG. 33A is a graph showing results for Hb modification in healthy blood incubated with PenLab compounds for 1 hour as indicated. FIG. 33B is a set of HPLC tracings showing the analysis.

FIG. 34 is a graph showing the % RBC lysis of control blood (combined).

FIG. 35A through FIG. 35F show data related to deformability (LORRCA, healthy controls); 0.95 Pa (FIG. 35A), 1.69 Pa (FIG. 35B), 3 Pa (FIG. 35C), 5.33 Pa (FIG. 35D), 9.49 Pa (FIG. 35E), 30 Pa (FIG. 35F).

FIG. 36A through FIG. 36D show results for EIₘᵢₙ (FIG. 36A), EIₘₐₓ (FIG. 36B), EI_{delta} (FIG. 36C), and EI_{hyper} (FIG. 36D) in healthy controls. FIG. 36E through FIG. 36H show results for osmolarity as indicated at 0 min (FIG. 36E), 0 EIₘₐₓ (FIG. 36F), 0 EIdelta (FIG. 36G) and 0 hyper (FIG. 36H).

FIG. 37A and FIG. 37B are graphs showing results for the oxygen dissociation curve (ODC).

FIG. 38A is a graph showing the modified Hb %. FIG. 38B is a set of HPLC showing the traces of HMF prodrug and carbon dots.

FIG. 39 is a graph showing the % RBC lysis of SS blood.

FIG. 40A and FIG. 40B are graphs showing results for the oxygen dissociation curve (ODC).

FIG. 41A through FIG. 41D show results for POS 1^{st} derivative (FIG. 41A), EIₘᵢₙ (FIG. 41B), EIₘₐₓ (FIG. 41C), and delta EI (FIG. 41D). FIG. 41E through FIG. 41H show data: area (FIG. 41E), area between curves (FIG. 41F), POR 1^{st} derivative (FIG. 41G), and recovery (FIG. 41H).

FIG. 42 presents data on lysis of sickle cell blood cells for 1 or 3 hours with the indicated compounds.

FIG. 43A, FIG. 43B, FIG. 43C, and FIG. 43D present data for HEMOX (sickle cell).

FIG. 44A, FIG. 44B, FIG. 44C, and FIG. 44D present data for HEMOX (sickle cell; Hill number).

FIG. 45 presents LORRCA data, including point of sickling (FIG. 45A), point of recovery (FIG. 45B), area between curves (FIG. 45C), EImax (FIG. 45D), EImin (FIG. 45E), and Delta EI (FIG. 45F).

FIG. 46 presents data on mouse pharmacodynamica (B6 Control Mice).

FIG. 47 is a graph showing RBC lysis (%) after 1- or 3-hour incubation as shown.

FIG. 48A through FIG. 48H present data related to osmotic fragility: EIₘᵢₙ (FIG. 48A), EIₘₐₓ (FIG. 48B), EI_{delta} (FIG. 48C). EI_{hyper} (FIG. 48D). 0 min (FIG. 48E), 0 EIₘₐₓ (FIG. 48F). 0 delta (FIG. 48G) and 0 hyper (FIG. 48H).

FIG. 49 is a western blot, showing a molecular weight ladder and three lanes as discussed in Example 17.

FIG. 50A and FIG. 50B are images of sickle cells (SS) fixed at Oxy (150 Torr) and control fixed RBC at about 4 Torr, respectively.

### DETAILED DESCRIPTION OF EMBODIMENTS OF THE INVENTION

### 1. Overview

We disclose here nano-enabled delivery of highly potent 5-hydroxymethyl furfural (5-HMF) to sickled blood cells. This unique approach involves a ‘dual sourced’ agent for HMF delivery. Sustained and safer delivery of drugs can be achieved through a well-defined nanoparticle platform, which can assist in overcoming the previously encountered shortcomings for hydroxyurea-based drug delivery. Current delivery approach for HMF is far from being ideal, with modestly enhanced efficacy but additive toxicity exerted by the agent delivered. Typically, the free drugs get distributed in the body in a non-specific manner, thus producing adverse side effects. Articulate design of a well-defined nanostructure allows integration of both the therapeutic agents with varying polarity profiles, avoidance of systemic dis-integration, and reduction of individual drug related toxicity. The properties of this system also include longer systemic circulation and ability for sickle RBC accumulation through active target-specific mechanism.

Potent and specific anti-sickling effect has been demonstrated using 5-membered aromatic scaffold, 5-hydroxymethyl furfural (5-HMF). Results have indicated that 5-HMF possesses all the unique properties of a potential drug candidate and clearly warrants further preclinical evaluation.

The safety and efficacy of 5-HMF can be significantly elevated by using a targeted nanomedicine approach. Systemic delivery of the chemotherapeutic agent in a ‘protected’ prodrug nanoparticle form, by virtue of requiring a lower administered dose, reduces systemic toxicity while simultaneously enhancing drug uptake in the sickle RBCs.

According to embodiments of the invention, HMF-phospholipid prodrug shell and a carbon nanoparticle-based core form a particle. Sucrose-derived carbon nanoparticles metabolically degrade to HMF byproducts. Phospholipid-based prodrug-coated carbon nanoparticles essentially provide two sources of the active form of the drug, i.e., HMF. Sickle RBCs are known to have a high concentration of lipases and in the presence of such an enzymatic reservoir, lipases activate the phospholipids prodrug to release the active form of the HMF. Simultaneously, the carbon core undergoes metabolic degradation to HMF. So, in principle, the dual release mechanism works in sync and releases the drug with slow (prodrug) and fast kinetics.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the terms “administer,” “administration,” and their cognates refers to contacting a subject with a therapeutic compound or composition. As used herein, “administering” and its cognates refers to introducing an agent to a subject, and can be performed using any of the various methods or delivery systems for administering agents and pharmaceutical compositions known to those skilled in the art.

As used herein, the terms “treatment,” “treating,” and the like, refer to obtaining a desired pharmacologic and/or physiologic effect. “Treatment,” therefore includes: (a) preventing, delaying, or reducing the likelihood of developing the condition or disease or symptom thereof from occurring in a subject which may be predisposed to the condition or disease but has not yet been diagnosed as having it; (b) inhibiting the condition or disease or symptom thereof, such as, arresting its development; and (c) relieving, alleviating or ameliorating the condition or disease or symptom thereof, such as, for example, causing regression or lessening of the condition or disease or symptom thereof.

As used herein, the term “therapeutic agent” refers to any compound that exerts a “therapeutic effect” or pharmaceutical composition that contains such a compound. A “therapeutic effect” is a pharmacological or physiological effect of preventing, curing, delaying, ameliorating, improving, shortening the duration of, decreasing the likelihood of, decreasing the symptoms of, and the like, of a disease or condition in a subject.

As used herein, the term “therapeutic amount” or “therapeutically effective amount” means an amount that achieves the intended therapeutic effect in a subject. The full therapeutic effect does not necessarily occur by administration of one dose and may occur only after administration of a series of doses. Thus, a therapeutically effective amount may be administered in one or more administrations per day for successive days.

As used herein, the terms “subject,” “individual,” “host,” and “patient,” are used interchangeably to refer to any animal, and can include simians, humans, avians, felines, canines, equines, rodents, bovines, porcines, ovines, caprines, mammalian farm animals, mammalian sport animals, and mammalian pets. A suitable subject for the invention preferably is a human that is suspected of having, has been diagnosed as having, or is at risk of developing a sickle cell disease or condition. Conditions amenable to treatment by the invention which define a appropriate subject or patient will be discerned easily by the person of skill in the art based on the disclosures herein.

As used herein, the term “sickling” refers to the development of sickle erythrocytes in the blood; the term “antisickling” refers to preventing or counteracting the sickling of erythrocytes.

As used herein, the term “sickle cell disease (SCD)” refers to a group of mostly inherited blood disorders that includes including sickle cell anemia, HbSS, HbSC, Hb S beta-thalassemia, and the like.

As used herein, the term “subject in need” refers to a subject who suffers from or is likely to be suffering from any sickle cell disease.

As used herein, the term “point of sickling (POS)” quantitatively and reproducibly describes the defined loss of deformability of SCD RBCs. As used herein, the term “point of recovery (POR)” is defined as inhibition of sickling.

As used herein, the term “nanotherapy” or “nanomedicine” refers to medical treatment or therapy involving the use of nanotechnology, including nanoparticles. Nanotechnology typically deals with dimensions and tolerances of less than about 100-200 nanometers (nm). With respect to the present invention, the nanoparticles generally are in the range of about 0.1 nm to about 200 nm, or more preferably of about 1 nm to about 100 nm, and most preferably about 50 nm to about 100 nm.

As used herein, the term “prodrug” in the context of this invention relates to a lipid complexed to the sugar 5-HMF to form a “ball,” wherein the connection between the lipid and the sugar is designed to be cleaved by PLA2. The “prodrug lipid nanoparticle (NP)” is a prodrug lipid plus phosphatidylcholine (PC) mixed in a ratio of about 1:1 to form liposomes. The “prodrug lipid carbon dot (CD)” is an NP with a carbon dot core. A carbon dot nanoparticle (CDNP)” contains no lipid, but only the carbon dot core, as a control for the CD.

As used herein, the term “nanoparticle” refers to amorphous carbon nanoparticles.

As used herein, the term “nanoparticle pharmaceutical composition” refers to a nanoparticle with a pharmaceutically acceptable carrier to form a composition amenable to administration to a subject in need. Such compositions include solutions, suspensions, emulsions, topical formulations, transdermal formulations, and the like.

As used herein, the term “prodrug” refers to a compound with little or no pharmacological activity that metabolizes inside the body and converts into a pharmacologically active drug compound by various endogenous trigger.

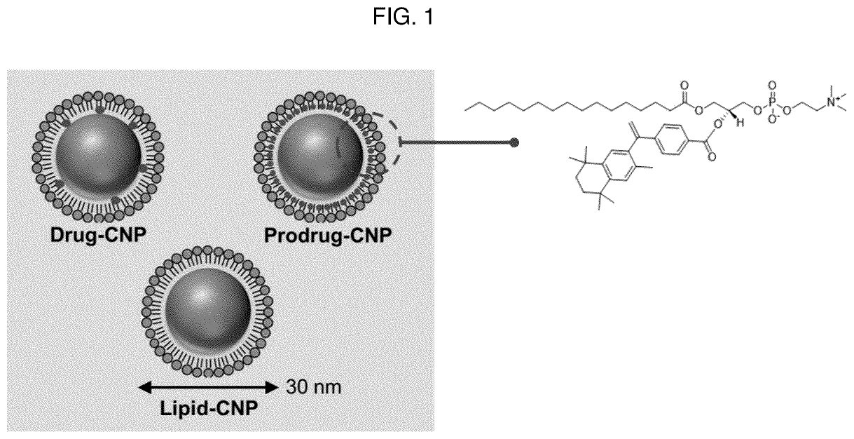

As used herein, the term “carbon dot” refers to a carbon nanoparticle such as graphene quantum dots, carbon nanodots, carbonized polymeric dots, and the like as known in the art. The carbon dots optionally comprise surface functional groups. Preferred carbon dots useful for this invention are nanoparticles derived from carbonization of sucrose. Carbon dots (CDs) are a class of photoluminescent nanomaterials with characteristics including good aqueous solubility, low toxicity, biocompatibility, ease of synthesis, fluorescence stability, eco-friendliness, and abundance of functional groups on their surface for ligand conjugation. See FIG. 1.

### 3. Embodiments of the Invention

### A. Introduction

Allosteric Hb-adducts that destabilize deoxyHbS target the mechanistic trigger for SCD pathology. 5-hydroxymethylfurfural (5-HMF) binds covalently to the Hb-NH2-terminal-αValine1 of hemoglobin, increasing oxygen affinity, reducing sickling and protecting hypoxic SS mice (survival, organ injury). However, 5-HMF (Aes-103) did not advance beyond PhI/II human trials, due to pharmacokinetic (PK) limitations (half-life ˜1 h, low bioavailability). A related (less potent) molecule, Voxelotor, was approved based on surrogate endpoints (Hb level and hemolytic markers), but without reduced vaso-occlusive morbidities.

In the present invention, 5-HMF, a superior antisickling agent, has been reformulated to address translation limitations arising from disfavorable pharmacokinetics. Specifically, we created two complementary 5-HMF prodrugs that are integrated as a ‘protected’ biocompatible composite nanoparticle designed to readily fuse with RBCs and be amenable to transdermal delivery (5-HMF-grafted-phospholipid layered over a sucrose-derived graphitic carbon dot core). The prodrugs of the invention release 5-HMF through complimentary mechanisms and rates (acutely from the shell by PLA2 and slowly/sustained from the core by enzyme-triggered and peroxide-based degradation).

Using human SS RBCs, the time- and concentration-dependent in vitro pharmacodynamic parameters for these prodrug nanoformulations (alone/combined) were measured. Here 3-hour data is reported for the composite NP: Hb-5-HMF modification (HPLC, 76.7±8.9%), HbO₂ affinity (p50 15.4±3.8 Torr) and antisickling potency (LORRCA deformability/elongation index D0.28±3.8.07AU & point of sickling D⁻ 21.4±8.2 Torr); imaging: ˜75%⁻ hypoxia-induced sickling (p<0.05, ANOVA, all). In vivo validation can be performed in Townes SCD mice, to define PK/PD and to determine benefit (morbidity, mortality) during hypoxic stress.

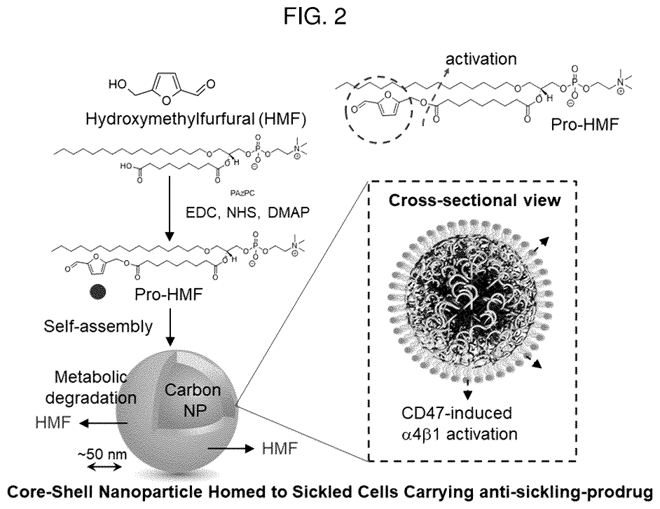

The invention provides an approach which enhances the therapeutic effect and safety of these agents by the virtue of a combination of prodrug approach with enhanced nano-delivery. See FIG. 2, which provides a general diagram of the composition for delivery of anti-sickling (e.g., 5-hydroxymethyl furfural) or other drug compounds. The overarching goal of this invention is to bring a much-needed nanomedicine approach for the delivery of HMF for SCD. The delivery of the drug is mediated by a dual prong approach that covers metabolic degradation and phospholipids prodrug activation.

### B. 5-HMF

SCD results from a single-point mutation in hemoglobin (Hb), in which βGlu6 is replaced by Val in sickle Hb (HbS). Upon deoxygenation, HbS polymerizes, causing red blood cell sickling. Sickled RBCs impair blood flow, with cascading adverse effects that lead to chronic organ damage, poor life quality, and decreased life expectancy. Therefore, prevention of HbS polymerization and RBC sickling is a focus for SCD therapeutic development. During circulation, sickling is promoted by HbO₂ release.

Hydroxymethylfurfural (HMF), also known as 5-(hydroxymethyl)furfural (5-HMF), is a chemical compound that inhibits the formation of sickled cells in blood and is being considered for the treatment of sickle cell disease. The structure of HMF is provided below.

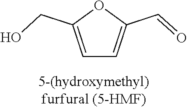

5-HMF is an allosteric effector of hemoglobin which increases its affinity for oxygen. It has been studied in patients with sickle cell disease for its antisickling effects, seen in vitro and in vivo. In addition, its ability to increase NO bioavailability can mitigate the severity of SCD symptoms. However, the poor pharmaceutical characteristics of 5-HMF have resulted in a cessation of studies of this drug for SCD treatment.

5-HMF forms covalent Schiff-base interactions with α-subunit N-terminal αVal1 amines in the Hb α-cleft that increase HbO₂ affinity. 5-HMF also appears to directly inhibit T-state HbS polymerization by weakening intermolecular contacts critical to fiber stability. When incubated with SRBCs, 5-HMF inhibits hypoxia-induced sickling, rheological impairment and SRBC dehydration in a dose-dependent manner, In Townes SCD mice, in vivo, 5-HMF protects against hepatic vaso-occlusion and lethal hypoxia. In this invention, 5-HMF has been formulated to address translation limitations arising from disfavorable pharmacokinetics.

Specifically, two complementary 5-HMF prodrugs were integrated into a protected biocompatible composite nanoparticle (5-HMF CompNP) that is designed to readily fuse with RBCs and be amenable to transdermal delivery, for example as a 5-HMF-grafted-phospholipid layered over a sucrose-derived graphitic carbon dot core. These prodrugs were demonstrated to release 5-HMF through complimentary mechanisms and rates (acutely from the shell by PLA2 and slowly/sustained from the core by enzyme-triggered and peroxide-based degradation). Using human SRBCs, time- and concentration-dependent in vitro pharmacodynamic parameters and antisickling potency were determined for these prodrug nano-formulations (alone/combined).

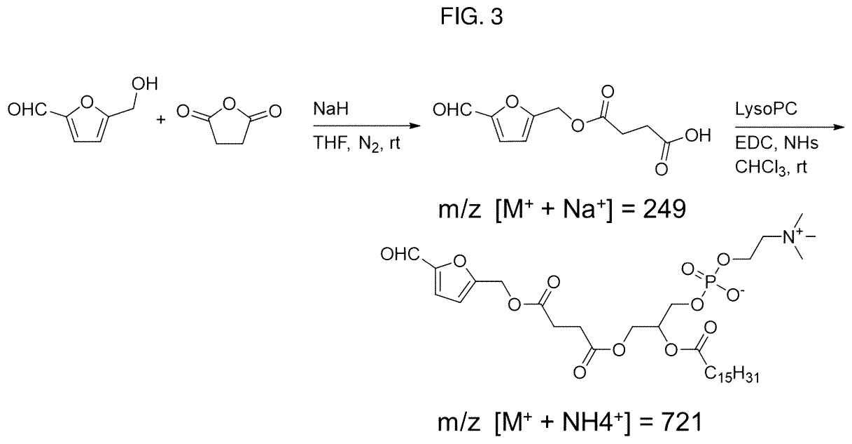

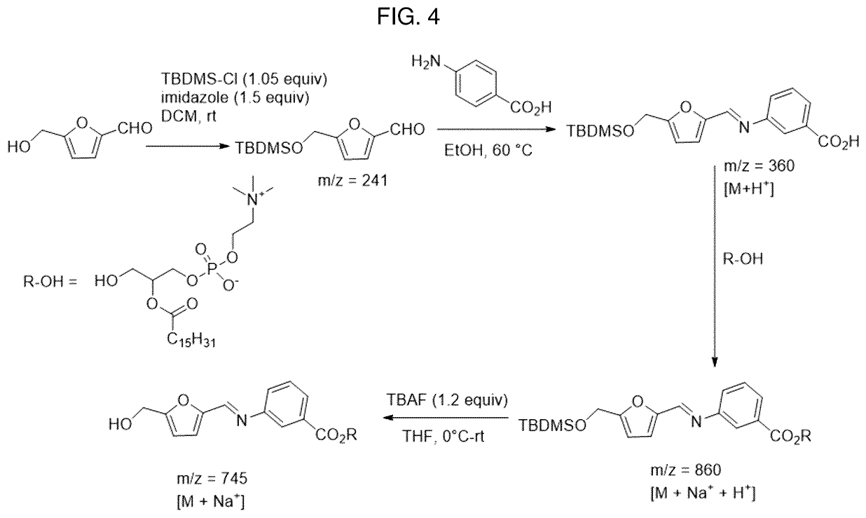

5-HMF phospholipid prodrugs are synthesized as shown in FIG. 3. An alternative synthetic route is shown in FIG. 4.

### C. Nanoparticles

Two important advantages of this innovative approach merit comment. First, pro-drug nanoparticle formulations offer remedies to PK/PD flaws that limit translation of otherwise potent/safe active compounds, by virtue of protection from enzyme degradation, prolonged half-life, improved targeting, and ability to achieve sustained release. The formulation of this invention also can serve as a ‘swappable’ platform for delivery of other optimized 5-HMF derivatives, other drugs, or for a combination treatment platform for multiple or additional drugs that can be incorporated into the LP prodrug that is layered over the CD core.

Second, this approach is readily amenable to microneedle (MN patch) transdermal delivery—bypassing the gastrointestinal system and improving compliance by replacing (more frequent) hypodermic injection with weekly patch changes. In addition to use for ‘simple’ compounds (levonorgestrel, insulin, calcitonin, etc.), MN patches have been used for nanocrystals (pure drugs), lipid-based and polymeric nanoparticles (drug/prodrug payloads) and inorganic nanoparticles (e.g., silicas, metals) to treat a variety of indications, such as skin cancer, diabetes, cardiovascular diseases, or contraception.

The nanoparticles preferably are about 0.1 nm to about 200 nm in diameter, more preferably about 1 nm to about 100 nm in diameter, and most preferably about 50 nm to about 100 nm in diameter. The nanoparticles were produced as described in the Examples below. In summary, the lipid is complexed to the sugar 5-HMF to form a prodrug-lipid ball. The connection between the lipid and the sugar is designed to be cleaved by PLA2. The prodrug lipid plus phosphatidylcholine mixed together in a ratio of about 1:1 to form liposomes is termed the “prodrug lipid nanoparticle.” A “prodrug lipid carbon dot” is a prodrug lipid nanoparticle which has a carbon dot core. A “carbon dot nanoparticle” is a control particle comprising a carbon dot core with no prodrug lipid.

### D. Dosage Forms

The compounds of the invention include the base, and any pharmaceutically acceptable hydrate, solvate, acid or salt, of 5-HMF or other drug, in the form of nanoparticles as described herein. In preferred embodiments, the nanoparticles described herein are formulated and are administered as a pharmaceutical composition that includes a pharmaceutically acceptable carrier and the inventive nanoparticles, and may also optionally include one or more additional pharmaceutical agents, including optionally 5-HMF.

A pharmaceutically acceptable carrier refers to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art.

A suitable pharmaceutical carrier typically depends on the intended route of administration for the therapeutic modality. A non-inclusive list of carriers and vehicles contemplated for use with the invention include, but are not limited to fillers, adjuvants, pH adjusters, solvents, transdermal enhancers, dispersing agents, suspending agents, emulsifiers, wetting agents, lubricants, preservatives, adhesives, bandages or coverings, transdermal patches (and elements thereof), and containers (e.g., ampoules, bottles, pre-filled syringes, envelopes, boxes, and the like). Persons of skill are well aware of components needed to produce a suitable transdermal form of pharmaceutical and how to construct such devices.

### E. Routes of Administration

A suitable carrier depends to a large degree on the route of administration contemplated for the pharmaceutical composition. Routes of administration are determined by the person of skill according to convenience, the health and condition of the subject to be treated, and the location and stage of the condition to be treated.

Such routes can be any route which the practitioner deems to be most effective or convenient using considerations such as the patient, the patient's general condition, and the specific condition to be treated. For example, routes of administration can include, but are not limited to parenteral administration including transdermal, transmucosal, oral, intravenous, intraarterial, intrathecal, subcutaneous, intradermal, intraperitoneal, rectal, vaginal, topical, nasal, sublingual, buccal, and inhalation, or local administration including with a wound covering or dressing, topical, direct injection into an area to be treated, and the like. The administration can be given by transfusion or infusion, and can be administered by an implant, an implanted pump, or an external pump, or any device known in the art. A preferred route of administration for the present invention is transdermal, preferably using a transdermal patch.

Therefore, the forms which the pharmaceutical composition can take will include, but are not limited to: transdermal patches, tablets, capsules, caplets, lozenges, dragees, pills, granules, oral solutions, powders for dilution, powders for inhalation, sterile solutions or other liquids for injection or infusion, buccal patches, inserts and implants, rectal suppositories, vaginal suppositories, creams, lotions, oils, ointments, topical coverings (e.g., wound coverings and bandages), suspensions, emulsions, lipid vesicles, and the like. Preferably, the nanoparticles of embodiments of the invention are delivered transdermally or by intravenous injection, and therefore will be provided in a transdermal patch or sterile solution, suspension, or emulsion suitable for injection.

### F. Subjects

The preferred subject contemplated for using the embodiments of the invention is a human subject with sickle cell disease or sickle cell trait. The subject may or may not currently have active sickle cell anemia, sickle cell crisis, or any overt symptoms. Any person genetically disposed to sickle cell disease or sickling of the red blood cells is contemplated to use embodiments according to the invention.

The diseases which are contemplated to benefit from treatment with the nanotherapy according to the invention include sickle cell anemia, Hb SS, Hb SC, Hb S beta-thalassemia, and the like.

### G. Methods of Treatment

Dosage amounts per administration include any amount determined by the practitioner, and will depend on the size/weight of the subject to be treated, the state of the health of the subject, the route of administration, the condition to be treated or prevented and its severity, and the like. In general, it is contemplated that for the majority of subjects, a dose in the range of about 0.01 mg/kg to about 100 mg/kg is suitable, preferably about 0.1 mg/kg to about 50 mg/kg, more preferably about 0.1 mg/kg to about 10 mg/kg, and most preferably about 0.2 mg/kg to about 5 mg/kg are useful. This dose can be administered weekly, daily, or multiple times per day. A dose of about 0.1 mg, 0.2 mg, 0.25 mg, 0.5 mg, 1 mg, 5 mg, 10 mg, 20 mg, 40 mg, 80 mg, 100 mg, 250 mg, 500 mg, or 1000 mg can be administered.

Treatment regimens can include a single administration or a course of administrations lasting two or more days, including a week, two weeks, several weeks, a month, two months, several months, a year, or more, including administration for the remainder of the subject's life. The regimen can include multiple doses per day, or one dose per day or per week, for example, or a long infusion administration lasting for an hour, multiple hours, a full day, or longer.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Hemox

Hemox analysis methods determine oxygen partial pressure and the degree of hemoglobin saturation with oxygen using a Clark oxygen electrode and spectrophotometry, respectively. From these data, one can produce an oxygen dissociation curve (ODC) and a Hill number (cooperativity).

### B. Laser-Assisted Optical Rotational Cell Analyzer (LORRCA) Methods

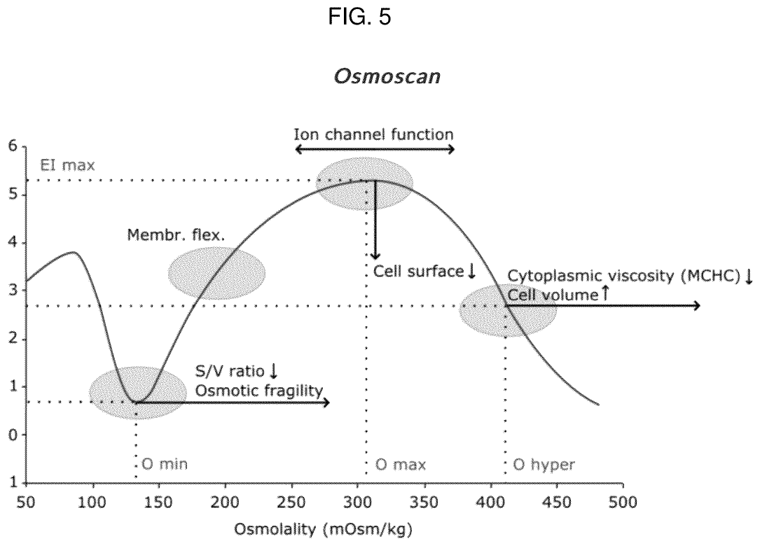

The LORRCA red blood cell analysis platform was used to obtain information on deformability (cell and cell membrane stability), including under an osmotic gradient (osmotic resistance). The platform also can provide data on aggregation tendency and kinetics and measure the relative oxygen pressure at the critical point when the red blood cells start to sickle. Here, RBC Osmotic Fragility (elongation index (EI) vs. osmolality at specific sheer), RBC Deformability (EI vs sheer at specific osmolality), and RBC Oxyscan Sickling Characteristics (EI vs pO₂) were determined. See, for example, FIG. 5.

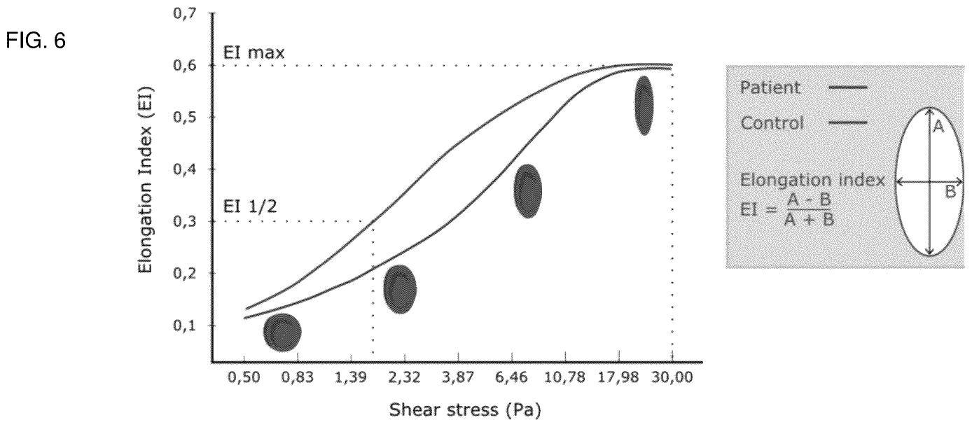

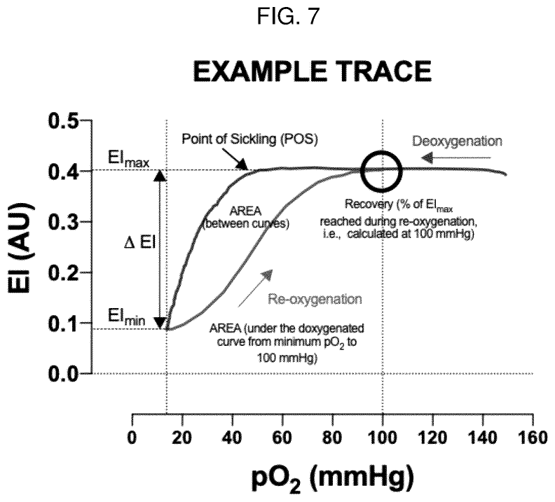

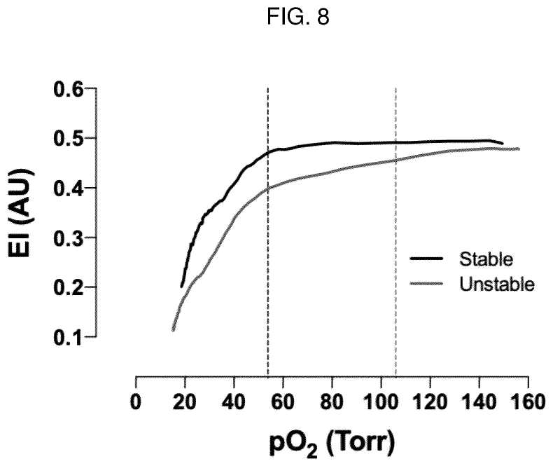

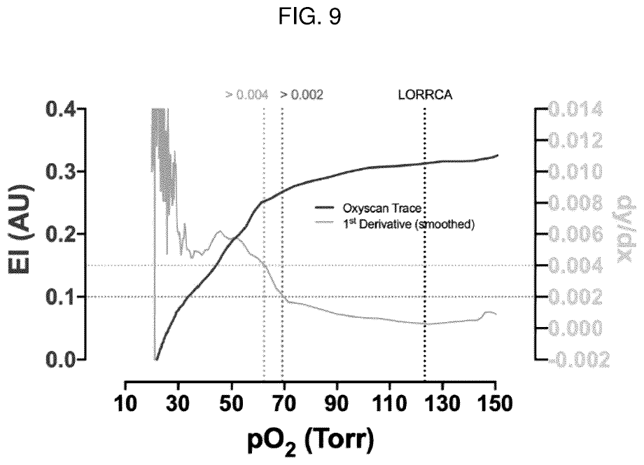

See FIG. 6 for a description of how EI is calculated. See FIG. 7 for an example calculation of Oxyscan analysis parameters. See FIG. 8 for a graph demonstrating the determination of the point of sickling by LORRCA. LORRCA determines POS as the pO₂ corresponding to a 5% decrease in EIₘₐₓ. If the EI is not completely stable at high pO₂ (i.e., the Oxyscan baseline is not level), POS is calculated to be artificially high. FIG. 9 is a graph showing an example point of sickling (POS) determination from “first derivative,” which is taken from the plot. A common method to process, the first derivative of a spectrum is the rate of change of absorbance with respect to wavelength.

### C. Confocal, Darkfield and Hyperspectral Imaging

Confocal, darkfield and hyperspectral imaging techniques were used. The images showed the spectral characteristic differences between HbA and HbSS.

### D. High Pressure Liquid Chromatography (HPLC)

HPLC was performed to detect binding of an allosteric effector to Hb.

### E. Western Blotting

Western blotting was performed as follows to determine PLA₂ activity in erythrocytes. Pelleted red blood cells were lysed in a buffer containing 50 mM TRIS-HCl, pH 8.9, 100 mM NaCl, 1 mM Na-orthovanadate. The total protein concentration was measured with Pierce™ BCA Protein Assay Kit (Life Technologies™) and PLA activity was measured by EnzChek™ phospholipase A2 Assay Kit (Life Technologies™).

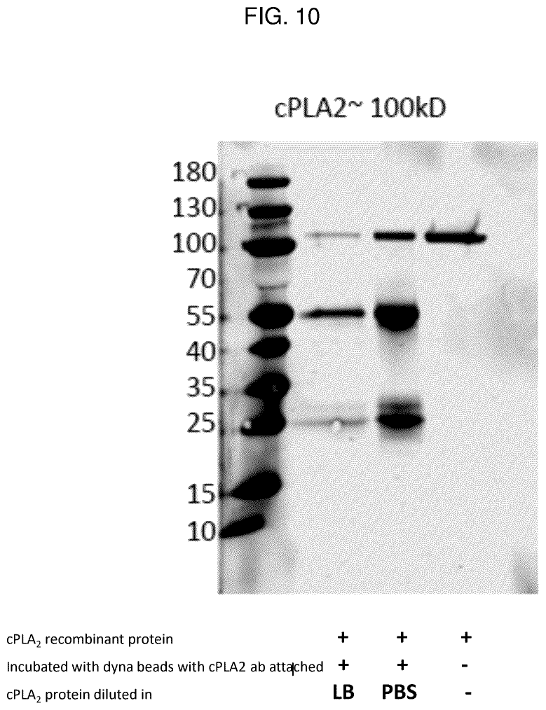

Lanes 1 and 2 contained 1 μL of recombinant cPLA2 diluted in 199 μL of either lysis buffer (LB) or phosphate buffered saline (PBS) for one hour at room temperature with DYNA beads. Lysis buffer contains Tris HCl, NaCl, KCl, glycerol, pH8 and is stored in aliquots at −80° C. The beads were washed twice with LB or PBS and stripped with 40 μL SDS loading buffer. Lane 3 contains 1 μL of recombinant protein put in 39 μL SDS loading buffer for 10 minutes at 95° C. The beads were removed and the samples loaded onto the gel as indicated. See FIG. 10.

The gel was probed with cPLA2 recombinant antibody (0.9 μg/μL) in TRIS HCl, sodium chloride, potassium chloride, and glycerol at pH 8 (stored at −80° C. in aliquots). See FIG. 10. Note that cPLA2 migrates to about 100 kDa on this gel. In FIG. 10, the left lane is a ladder with sizes as indicated. The next 3 lanes (lanes 1, 2, and 3) are as described, where all three lanes contained cPLA2 recombinant protein. For lanes 1 and 2, the protein was incubated with DYNA beads with cPLA2 antibody attached. The cPKA2 protein was diluted in LB (lane 1), PBS (lane 2), or not diluted (lane 3).

CPLA2 recombinant antibody (0.9 ug/ul) in Tris HCl/Sodium Chloride/Potassium Chloride/Glycerol, at pH 8 was stored at −80° C.; aliquoted prior to storage to avoid freeze/thaw). Here, lanes 1 and 2: 1 ul of recombinant cPLA2 diluted in 199 ul of either Lysis Buffer (LB) or PBS 1 hour RT with Dyna beads followed by washing beads ×2 with LB or PBS and stripping the beads with 40 μl SDS loading buffer. Lane 3: 1 μl of recombinant protein was put in 39 μl SDS loading buffer for 10 minutes at 95° C. The beads are removed and the sample loaded onto the gel.

### Example 2: Prodrug Nanoparticle Production

Two complementary 5-HMF prodrugs were developed: a phospholipid prodrug referred to as lipid prodrug or LP, and a graphitic prodrug referred to as carbon dot or CD. Each was evaluated individually and as an integrated self-assembled compound nanoparticle (CompNP).

The carbon dot (CD) cores were prepared by dissolving sucrose (200 mg) in 2 mL water (0.2 μM, 18 MΩ cm), heating at 270° C. for 30 minutes in a pressure confined reactor which produces the dots. The product is resuspended in 4 mL water and subjected to probe sonication (Q700, 20 min: Amp, 1, on: 2 second, off: 1 second), filtering (0.2 μm), and dialysis using a cellulosic membrane (10 kD MWCO) for 2-3 days. See Misra et al., Hyperspectral Imaging Offers Visual and Quantitative Evidence of Drug Release from Zwitterionic-Phospholipid-Nanocarbon when Concurrently Tracked in 3D Intracellular Space. Adv. Funct. Mater. 26:8031-8041, 2016 and Alafeef et al., Carbon Dots: from Synthesis to Unraveling the Fluorescence Mechanism. Small 2303937, 2023 for additional description.

CD biodegradation was ascertained via multiple physico-chemical characterization methods, including UV-visible spectroscopy, zeta potential, Raman and infrared spectroscopy, transmission electron microscopy, atomic force microscopy, nuclear magnetic resonance and mass spectroscopic techniques. Catalytic biodegradation of naked CDs was demonstrated via oxidative activity of pancreatic lipase and at low concentrations of hydrogen peroxide. Lipid-passivated CDs proceeded through the degradation of lipid molecules first, followed by degradation of CDs as a whole. In both cases, 5-HMF was a common metabolite.

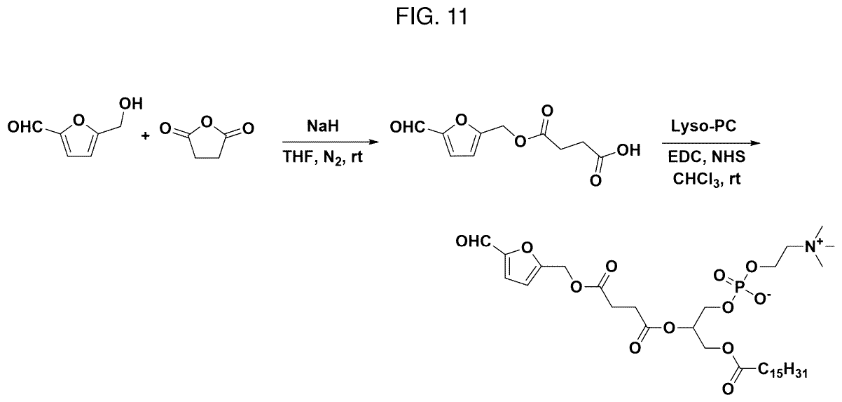

The phospholipid LP ‘pro-HMF’ compound (final structure shown in FIG. 11) was synthesized in two steps, as shown in FIG. 2 and characterized by ¹H-NMR and MS. 5-HMF was treated with NaH and tetrahydrofuran (THF) at room temperature under nitrogen, then mixed with 1-lysophosphatidylcholine (16:0 lysoPC) and 1-ethyl-3-(3-dimethylaminopropyl)-carbodiimide (EDC) and N-hydroxy-succinimide (NHS) (anhydrous chloroform at room temperature, ON). Graphitic CD ‘pro-HMF’ was synthesized as follows: sucrose in water (0.2 μM, 18 MΩ cm) was heated to 270° C. for 30 minutes, followed by sonication (Q700, 20 m: Amp, 1, on: 2 second, off: 1 second), filtering (0.2 μm) and dialysis (10 kD MWCO, 2 days). The carbon dots thus are made from sucrose followed by a coating with HMF prodrug.

Simple LNPs (comprised only of LP pro-5-HMF) were prepared by combining 5-HMF LP (5 mM) with L-α-phosphatidylcholine (1:1) in chloroform/methanol, then drying as a thin film that was kept under vacuum overnight, then resuspended (PBS), vortexed, rested at room temperature for 30 minutes, and probe sonicated (10 m: amp 1, on 2 seconds and off 1 second), then subjected to prolonged dialysis (2 days) against an infinite sink (PBS, pH 7.2).

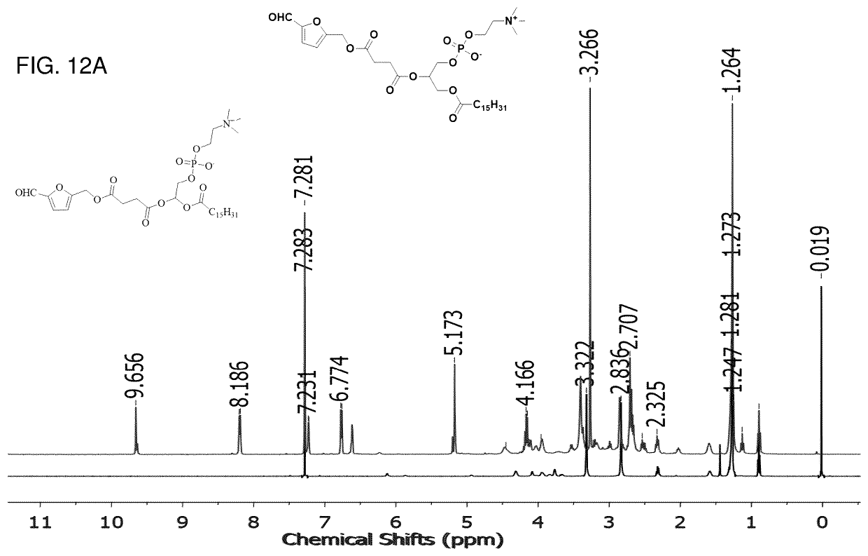

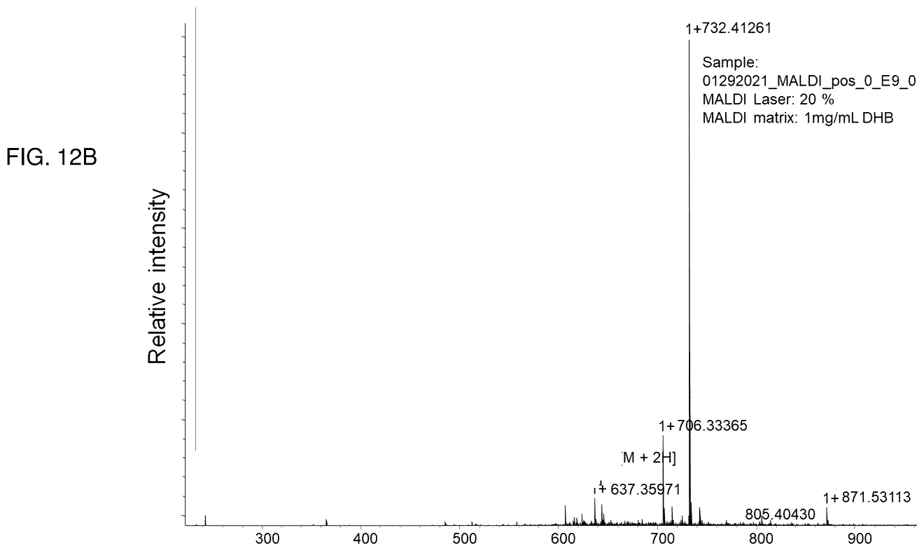

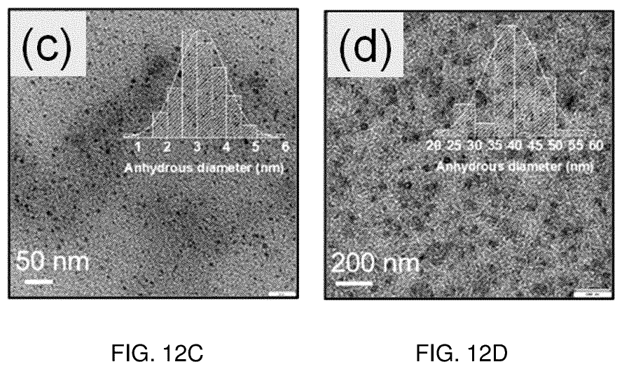

Compound Nanoparticle (CompNP) self-assembly occurred when CDs in PBS were mixed with the above lipid film, followed by sonication and dialysis as above. In this instance, for 20 mM prodrug lipid, the CD were made from 200 mg of sucrose, for a 1:7 molar ratio. The product can be produced with other ratios, for example 1:3, 1:4, 1:5, 1:6, 1:8, 1:9, or 1:10. A 5-HMF LP thin film was prepared as for our LNP formulation, which was then resuspended by mixing with a carbon dot suspension in PBS as prepared above and vortexing, then rested at room temperature for 20 minutes (see FIG. 2). The resulting mixture was then probe sonicated (10 minutes: amp 1, on 2 seconds and off 1 second), then subjected to prolonged dialysis (2 days) against an infinite sink (PBS, pH 7.2). The synthesized composite nanoparticles (CompNPs) were extensively characterized using multiple physico-chemical techniques (dynamic light scattering: 52 t 6 nm; electrophoretic potential: −39 mV) and TEM. See FIG. 12.

FIG. 2 presents the general scheme for synthesis of 5-phospholipid HMF-prodrug and the preparation of 5-HMF composite nanoparticles (CompNp). 5-HMF/NaH/tetrahydrofuran is mixed at room temperature for 12 hours, 72%; lyso-PC/1-ethyl-3-(3-dimethylaminopropyl) carbodiimide (EDC)/N-hydroxy-succinimide (NHS), and anhydrous chloroform are added at room temperature, overnight; then L-α-phosphatidylcholine (5 mM) 5-HMF lipid (5 mM) are mixed at a 1:1 molar ratio with carbon dots. This mixture was vortexed, left at room temperature for 30 minutes, then probe sonicated (10 m: amp 1, on 2 seconds and off 1 second), purified by dialysis 10 KDa MWCO for 2-3 days; then lyophilized and redispersed in 3 mL of 1×PBS. See FIG. 11 for the structure; see also FIG. 1, which depicts example nanoparticles.

FIG. 12 presents information on characterization of 5-HMF-prodrug and 5-HMF composite nanoparticles (CompNp). FIG. 12A shows overlaid ¹H-NMR spectra of 5-HMF-prodrug and lipid precursor to show successful synthesis (see also FIG. 11), FIG. 12B is a MALDI MS spectrum of the 5-HMF-prodrug; FIG. 12C and FIG. 12D present anhydrous state transmission electron microscopy (TEM) images of lipid-coated CDs and 5-HMF-prodrug-lipid coated CDs (carbon grid).

### Example 3: Nanoparticle Characterization

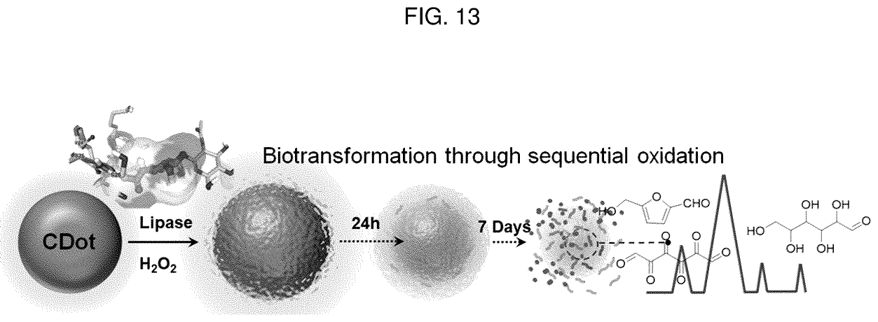

The synthesized composite nanoparticles (CompNPs) were extensively characterized. This process resulted in 152±12 nm (hydrodynamic size) particles with −34±6 mV surface charge. The nanoparticles undergo peroxide catalyzed degradation in the presence of lipase. See FIG. 13, which shows that enzymatic biodegradation in presence of H₂O₂ follows sequential oxidative pathway where intermediates are identified by MALDI-TOF spectroscopy. Testing revealed that the prodrugs release 5-HMF in RBCs through complimentary mechanisms (acutely from the LP shell by phospholipase A₂ (PLA₂) activity and slowly/sustained from the CD core by enzyme-triggered and peroxide-based degradation).

Using human SS RBCs, time- and concentration-dependent in vitro PK/PD parameters were obtained. Three-hour data for the composite NP included: Hb-5-HMF modification (HPLC, 76.7±8.9% 5-HMF modified HbS), HbO₂ affinity (p50 15.4±3.8 Torr) and antisickling potency (LORRCA deformability/elongation index Δ↑ 0.28±3.8.07 AU & point of sickling Δ↓ 21.4±8.2 Torr); imaging: ˜75%↓ hypoxia-induced sickling (p<0.05, ANOVA, all). Notably, PK (5-HMF modified SHb) for LNP & CompNP was sustained well beyond that for free 5-HMF and was progressive beyond 24 hours (likely due to ↑PLA2 and oxidizing milieu in SRBCs). PD (anti-sickling) for the CompNP formulation exhibited the greatest efficacy for (POS) (eg, ↑ hypoxia tolerance) and POR (simulating O₂ loading during pulmonary circulatory transit). In terms of PK data, the effect upon POS/POR progressed from 1 hour to 3 hours. Note that no produg/NP formulation resulted in hemolysis >1-2%.

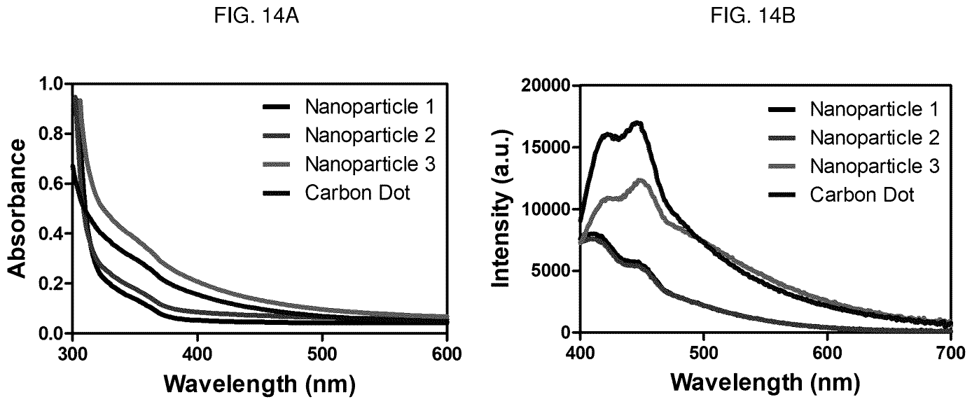

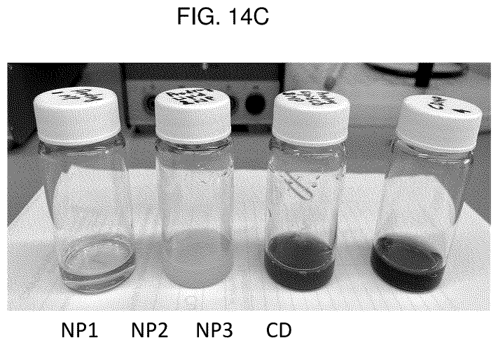

The following nanoparticles were produced and tested. See FIG. 14. Nanoparticle 1 was 5-HMF lipid at 10 mM concentration in 1×PBS; Nanoparticle 2 was L-α-Phosphatidylcholine (5 mM) 5-HMF lipid (5 mM) at 1:1 molar ratio; Nanoparticle 3 was L-α-Phosphatidylcholine (5 mM) 5-HMF lipid (5 mM) at 1:1 molar ratio with carbon dots (vortexed and left at room temperature for 30 minutes, probe sonicated (amp 1, on 2 seconds and off 1 second); purified by dialysis 10 KDa MWCO for 2-3 days; lyophilized and redispersed in 3 mL of 1×PBS; Nanoparticle 4 was carbon dots as prepared in 3 mL of 1×PBS 10 mM. See FIG. 14A and FIG. 14B, which show overlaid absorbance spectra and fluorescence spectra, respectively for carbon dot, prodrug lipid nanoparticle, prodrug lipid carbon nanoparticle and HMF prodrug. FIG. 14C shows the physical appearance of the nanoparticles.

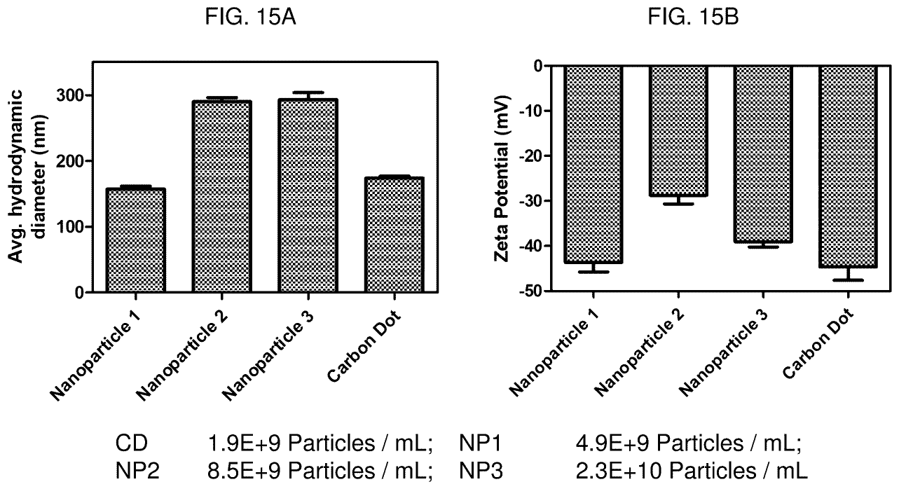

FIG. 15A and FIG. 15B are bar graphs showing the average hydrodynamic diameter and Zeta potential of the indicated nanoparticles.

### Example 4: Physico-Chemical Characterization

The particles exhibited unique degradation kinetics upon being subjected to enzyme oxidation.

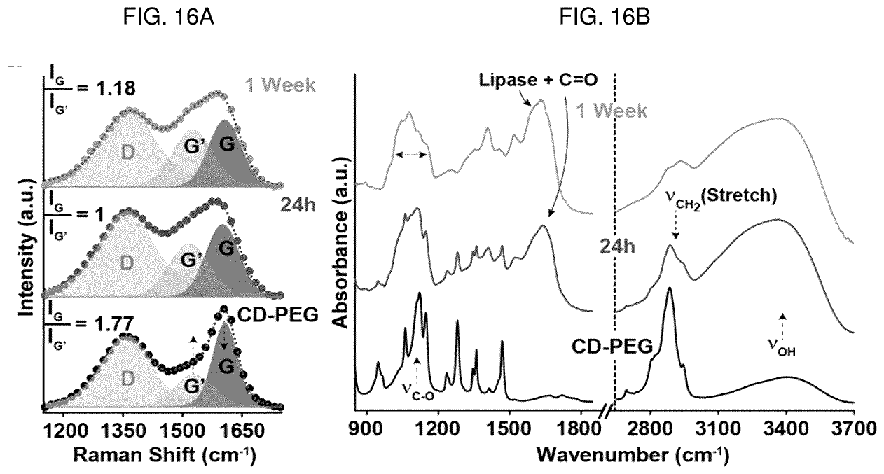

Further, this decomposition correlates with the relative accessibility of the enzymatic molecule. Using multiple physico-chemical characterizations, we have identified hydroxymethyl furfural as a metabolic by-product of these nanoparticles. FIG. 16A shows the Raman spectra of the CD-PEG samples in their pristine condition, subjected to lipase-peroxide oxidation for 24 hours and a week respectively. All spectra were fit to known D and G bands. The remaining contribution to the line shape was evaluated with the band G′. FIG. 16B shows the FTIR absorbance spectra of the particles. The broadening of the C—O (1000-1100 cm−1) modes, the presence of the amide I modes at 1650 cm⁻¹ and the increase in the O—H band intensity points to oxidation of the CD-PEG with lipase-peroxide system.

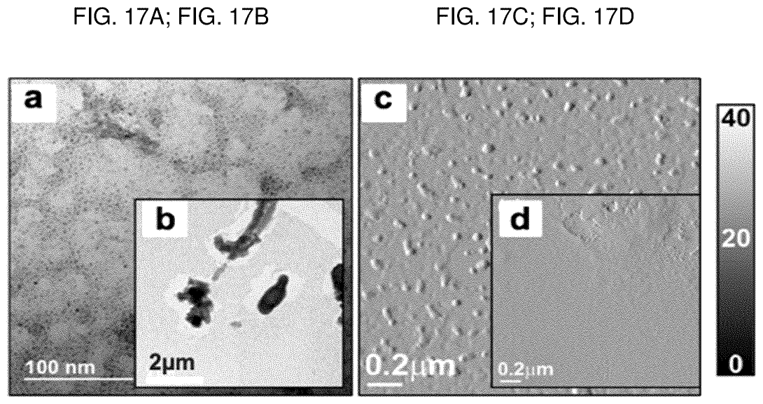

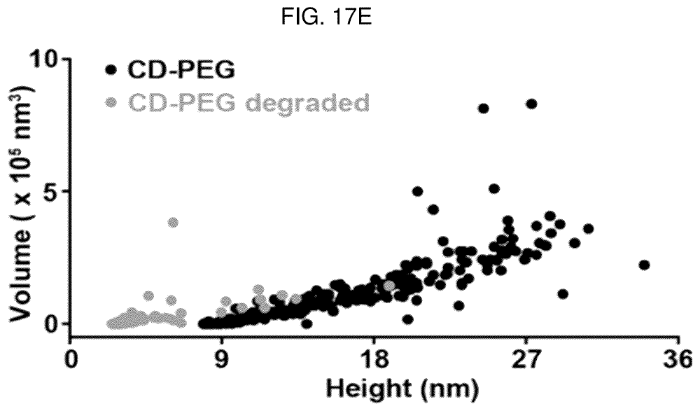

The effect of enzymatic degradation on morphological characteristics of CDs is shown in FIG. 17. Degradation was confirmed by morphological analyses using TEM and drop depositing pre- (FIG. 17A and FIG. 17B) and post (FIG. 17C and FIG. 17D) enzymatically treated samples. Biodegradation of CDs was monitored by atomic force microscopy (AFM), where CD-PEG was incubated with lipase and H₂O₂ at t=0 (FIG. 17C) and t=one week (FIG. 17D), respectively. A comparative analysis between circularity parameter and maximum height of nanoparticles (nm) obtained from AFM studies is shown in FIG. 17E. The data were plotted to relate between before and after degradation.

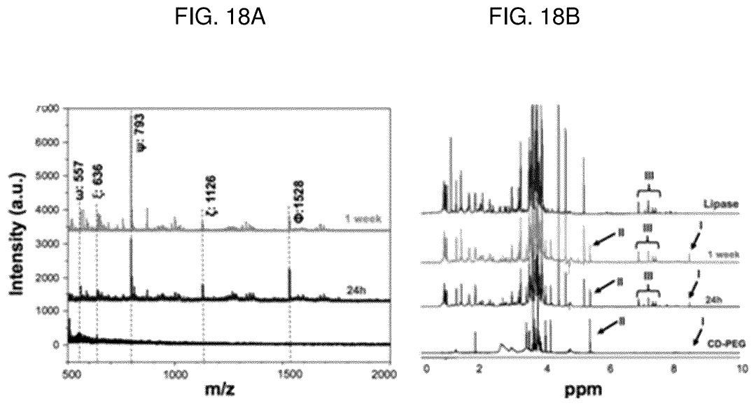

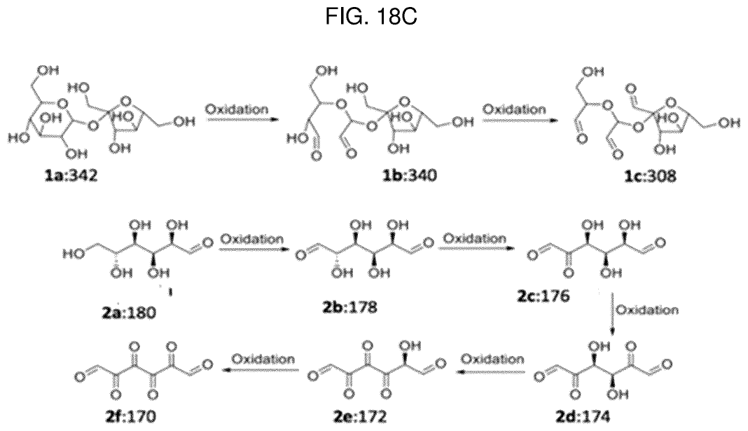

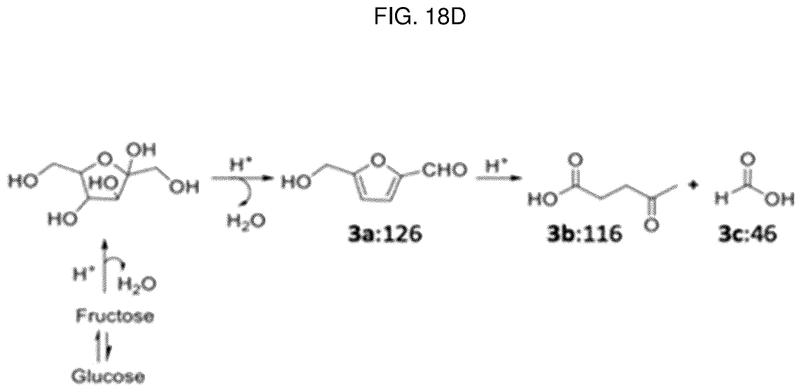

FIG. 18A shows the degradation of the nanoparticle monitored by MALDITOF spectrometry at different time points including after degradation for 0 hours, 24 hours, and 1 week. FIG. 18B shows the degradation of nanoparticles monitored by ¹H NMR spectroscopy. FIG. 18C shows the sequential oxidation process of sucrose and glucose leading to the formation of different intermediate products. FIG. 18D shows the degradation of sucrose in combined presence of Lipase and H₂O₂, leading to the formation of intermediate products. Corresponding m/z values are provided.

### Example 5: 5-HMF Pharmacokinetics (PK) and Pharmacodynamics (PD)

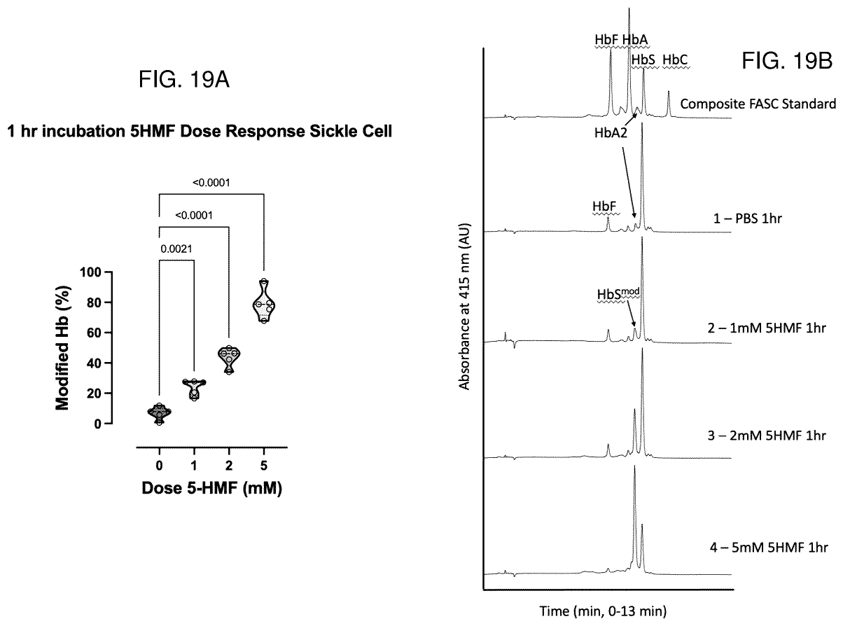

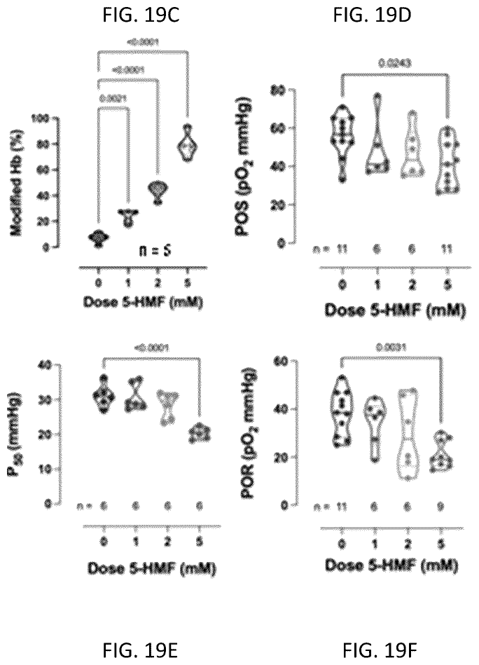

RBCs obtained from patients with SCD (Hct 20%) were incubated with 5-HMF (37° C., 1 hour) and hemolysates were prepared for cation exchange HPLC (Waters e2695XE Alliance, ProSwift WCX-1S PEEK). FIG. 19A presents illustrative chromatograms (standards & samples). FIG. 19B presents data concerning % 5-HMF modified Hb. FIG. 19C shows data concerning HbO₂ affinity (p50, O2 partial pressure at which Hb is 50% saturated). These data were determined in intact SRBCs (Hemox Analyzer) and demonstrate that about 75% 5-HMF˜Hb results in an about 35% increase in HbO₂ affinity. The effect of 5-HMF upon hypoxia-induced sickling was determined by laser ektacytometry during controlled SRBC deoxygenation (LORRCA Oxygenscan), which identifies the pO₂ at which RBC deformability maximally changes upon (FIG. 19D) deoxygenation (point of sickling, POS) and (FIG. 19E) reoxygenation (point of recovery (POR)).

### Example 6: 5-HMF Pharmacokinetics and Pharmacodynamics

RBCs from SCD patients were incubated with 5-HMF (20% Hct, 37° C., HMF: 0-5 mM, 0-3 hours). 5-HMF-induced Hb modification was quantitated by HPLC (Waters™ e2695XE Alliance™, Swift WCX-PEEK); O₂ dissociation curves (ODC) were measured in intact RBCs (HEMOX Analyzer); pO₂'s at the point of sickling (POS) and point of unsickling (POU) were determined during controlled Hb O₂ unloading/loading, respectively (LORRCA Oxygenscan RR Mechatronics). A dose dependent increase in 5-HMF induced Hb modification was observed. This increase was maximal (about 75%) at 1 hour and 5 mM 5-HMF concentration, and resulted in: (1) a 35% increase in HbO₂ affinity (ODC p50 pH 7.4; 31.1±3.1 Torr vs 20.3±1.5 Torr; 0 vs 5 mM 5-HMF), (2) a 30% POS decrease (56.9±10.1 vs 39.8±11.6 Torr; 0 vs 5 mM 5-HMF), and (3) a 45% POR decrease (38.3±9.1 vs 21.1±5.7 Torr; 0 vs 5 mM 5-HMF) (p<0.05 for all, RMANOVA).

### Example 7: Response with Incubation Time

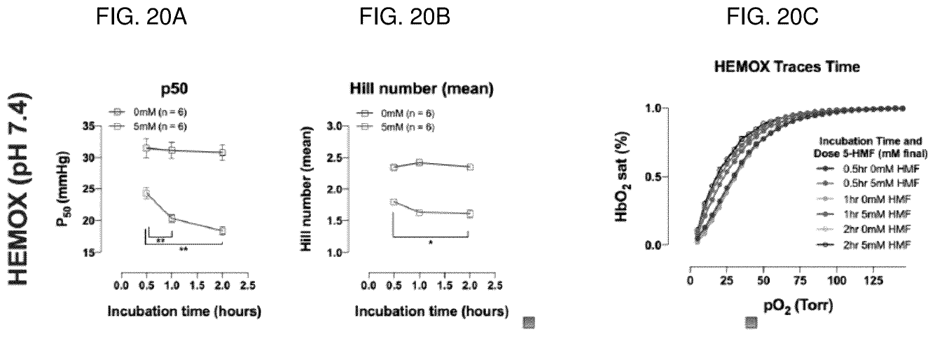

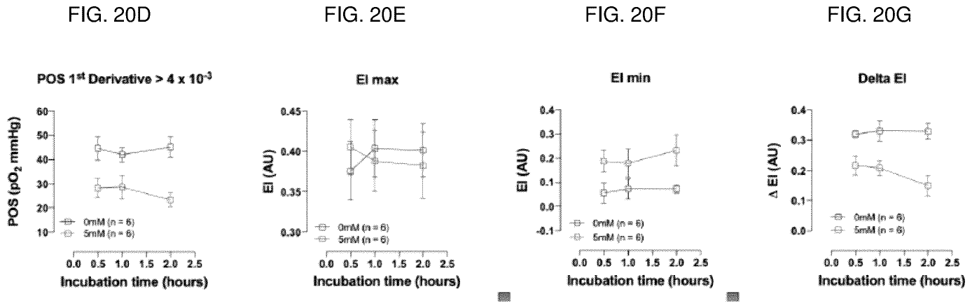

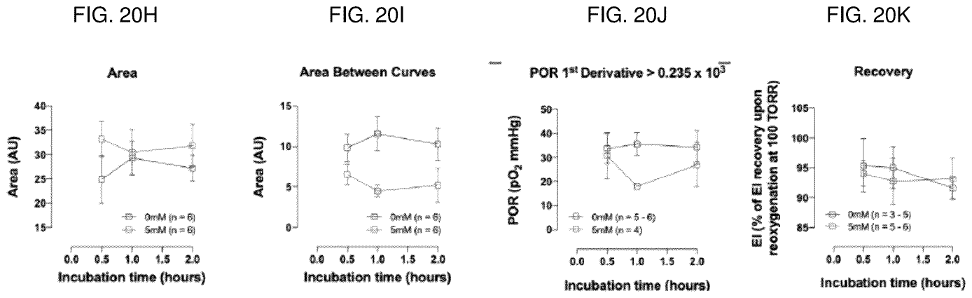

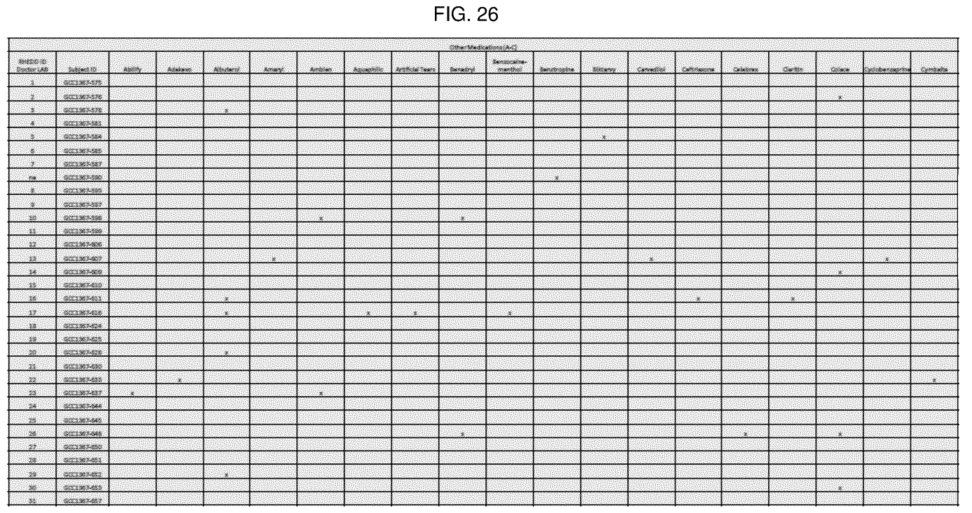

FIG. 20A through FIG. 20K are a set of graphs showing incubation time rcsponse. FIG. 20A (p50); FIG. 20B (Hill number (mean)); FIG. 20C (HEMOX traces time); FIG. 20D (POS 1^{st} derivative >4×10⁻³); FIG. 20E (EImax); FIG. 26F (EImin); FIG. 20G (delta EI): FIG. 20H (area); FIG. 20I (area between curves); FIG. 20J (POR 1^{st} derivative >0.235×10³); and FIG. 20K (recovery).

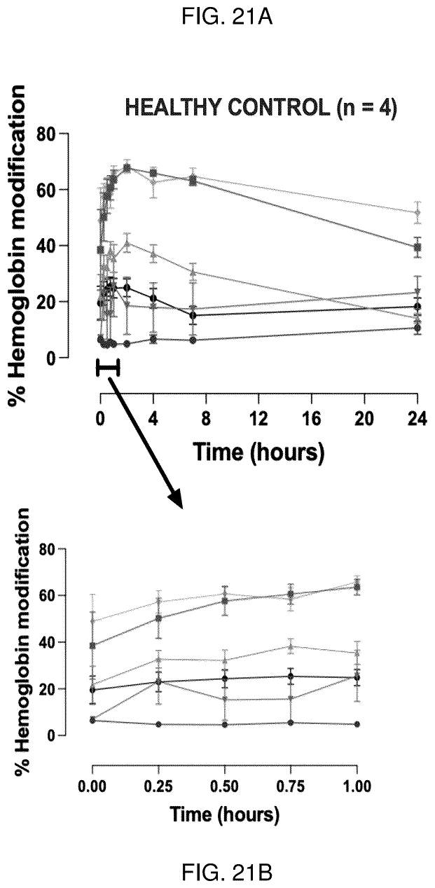

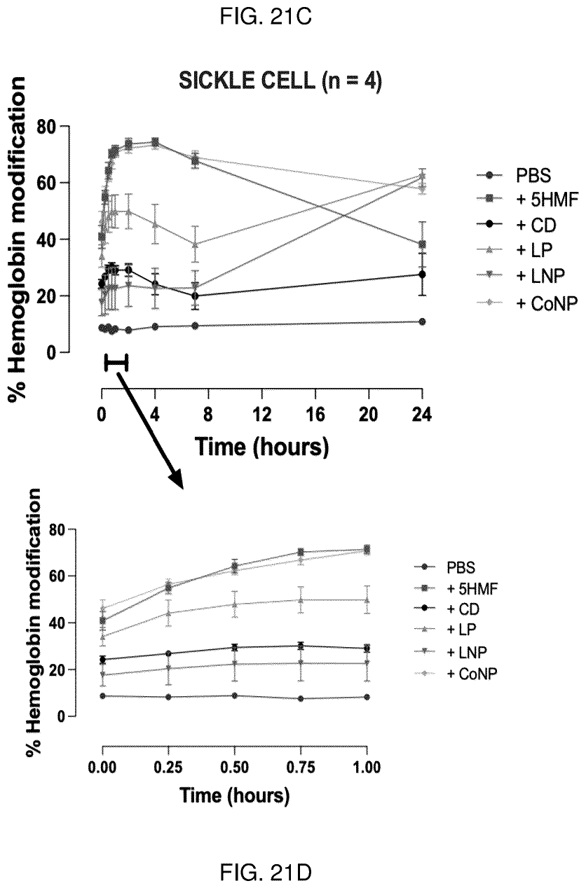

FIG. 21A and FIG. 21B (control patients) and FIG. 21C and FIG. 21D (SCD patients) present data on human RBCs.

### Example 8: 5-HMF Prodrug Pharmacokinetics

RBCs from controls, or SCD patients were incubated with PBS, 5-HMF, carbon dots (CD), 5-HMF lipid prodrug (LP), 5-HMF lipid prodrug nanoparticles (LNP), or 5-HMF composite nanoparticles (CoNP), all equimolar for 5-HMF (5 mM). See data in FIG. 21A and FIG. 21B (control patients) and FIG. 21C and FIG. 21D (SCD patients). Sequentially (at 0.25, 0.5, 0.75, 1, 2, 4, 6 or 24 hours) RBCs were washed and hemolysates were prepared for determination of % 5-HMF adducted Hb (as described above). Relative efficacy was (high to low): free CoNP, 5-HMF, LP, CD, LNP). Note that the effect for LP, LNP & CoNP is sustained beyond that of free 5-HMF; effect for LP, LNP & CD appears progressive beyond 24 hours (likely 2° to (known) ↑PLA2 and redox milieu in SRBCs).

### Example 9: 5-HMF Prodrugs and CNP Pharmacodynamics

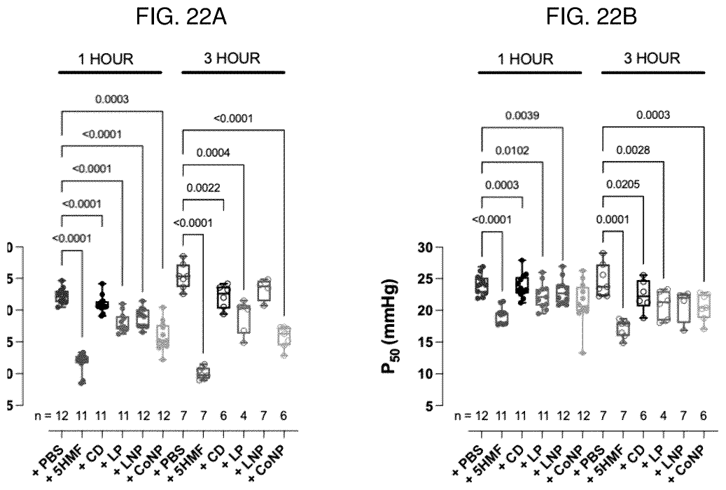

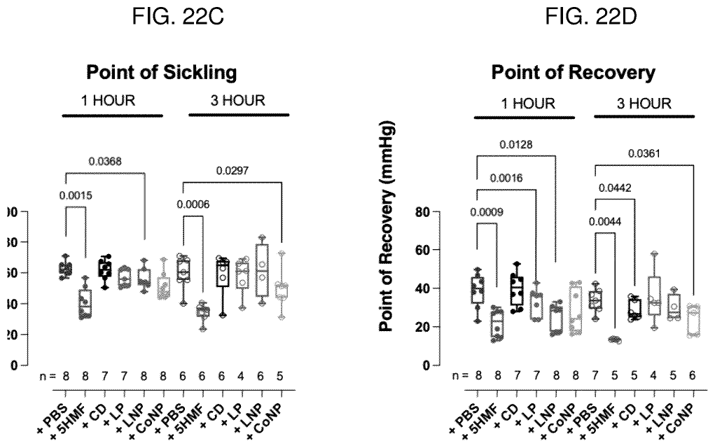

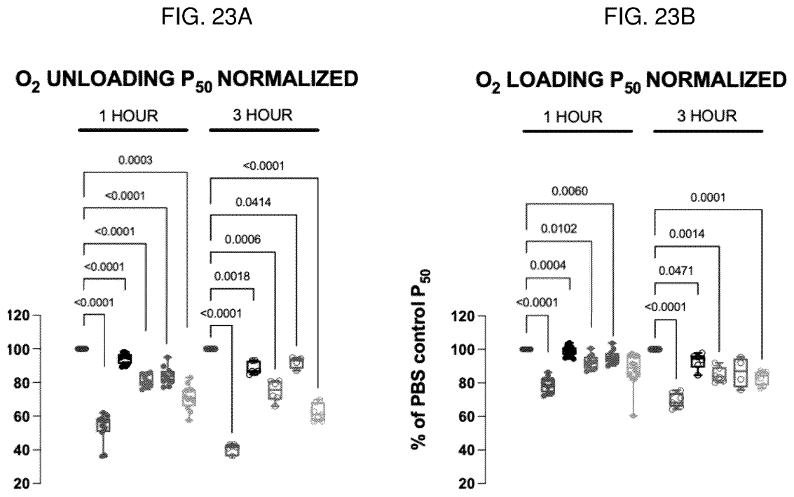

Human SCD RBCs were incubated with 5-HMF or 5-HMF prodrug formulations as described above with respect to FIG. 21 (n=6). After either 1 hour or 3 hours, either or both oxygen dissociation curves (ODC) or laser ektacytometry was performed to measure either or both p50 and POS/POR. Since the RBC O₂ unloading and loading curves exhibit hysteresis following 5-HMF incubation (due to differential effects upon R and T state Hb), p50 is presented for both ODC limbs. Consistent with PK data, the 5-HMF CompNP prodrug formulation exhibited the greatest efficacy with regard to p50 during (1) O₂ unloading (simulating systemic circulatory transit—see FIG. 22A), and correspondingly leading to a significantly lower pO2 for hypoxia induced point of sickling (POS) (e.g., improved hypoxia tolerance—see FIG. 22B). Similarly, the CompNP exhibited the greatest efficacy with regard to p50 during O₂ loading (simulating pulmonary circulatory transit—see FIG. 22C), and correspondingly leading to a significantly lower pO2 for reoxygenation induced point of recovery (POS) (e.g., HbS de-polymerization appears to initiate at lower pO2—see FIG. 22D). As for PK data, effect upon p50 and POS/POR appear to progress from 1 hour to 3 hours. Note that the no prodrug/NP formulation resulted in hemolysis >1-2%. See also the results in FIG. 23. CNP=carbon nanoparticle.

### Example 10: Patient Information

See Table 1, below, for patient characteristics.

TABLE 1
Patient Characteristics.

| | Sickle Cell (SS) | |
|---|---|---|
| Parameter | Blood (n = 6 donors ± SD) | |
| Mean Age, years (n) | 34.5 ± 14.0 | (29) |
| Gender, % male (n) | 60% | (29) |
| HbF # available | 16 | (29) |
| Mean WBC, thousands/μL (n) | 9.7 ± 3.9 | (29) |
| Hb, g/dL (n) | 9.8 ± 1.7 | (29) |
| HCT % (n) | 38.1 ± 5.3 | (29) |
| Platelet count (n) | 338 ± 126 | (29) |
| Neutrophils, Abs (n) | 5.6 ± 3 | (29) |
| Neutrophils, % (n) | 57 ± 11 | (29) |

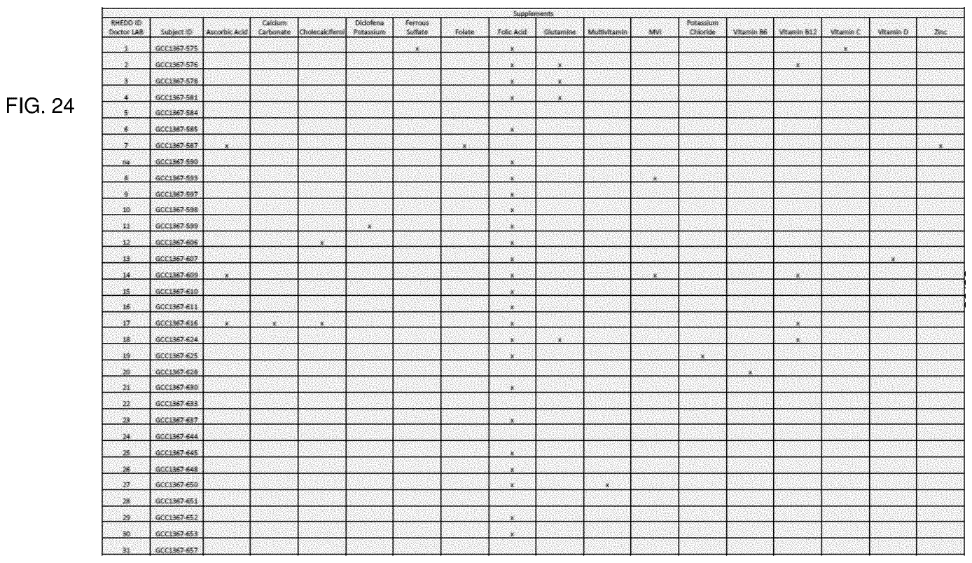

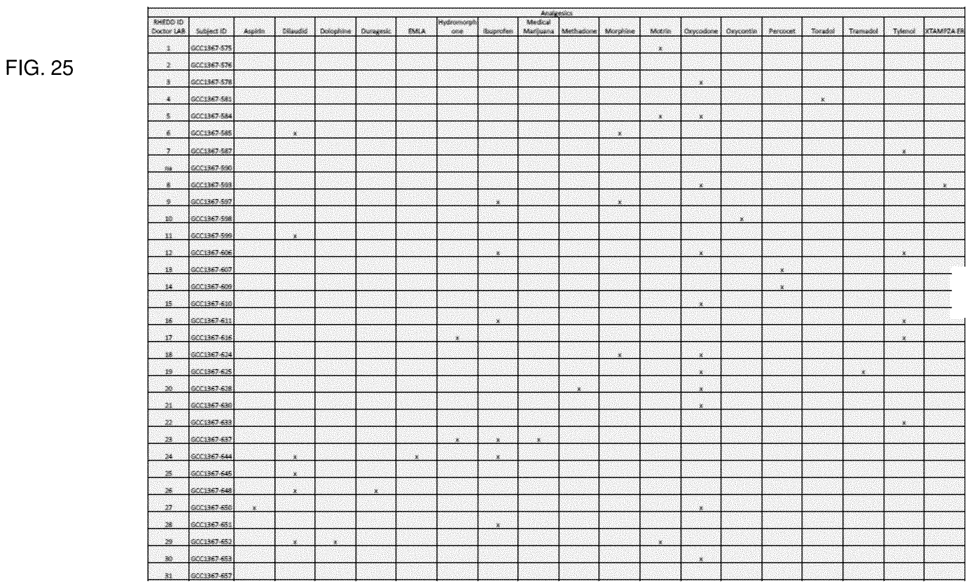

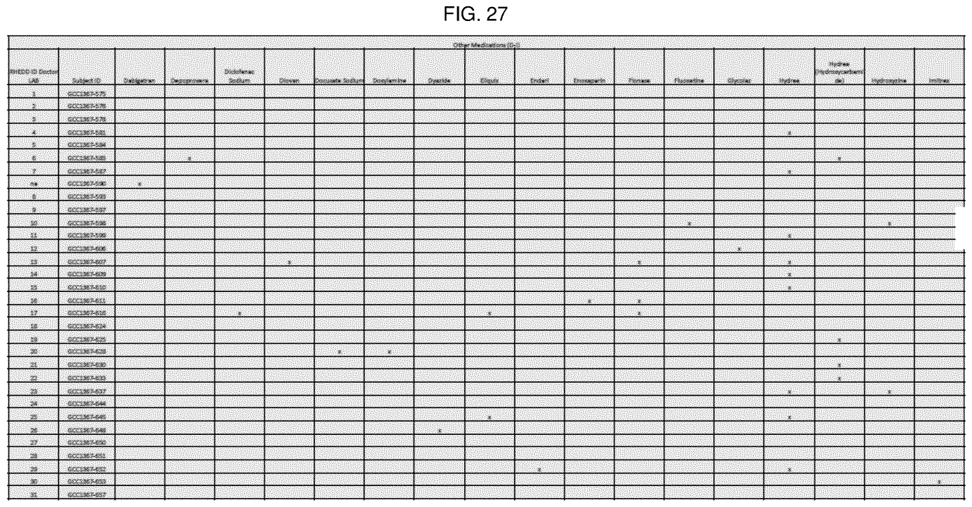

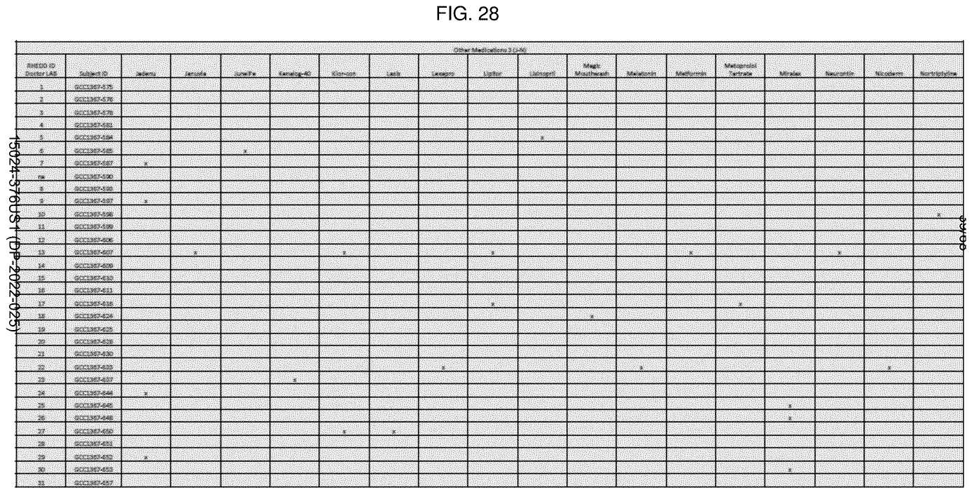

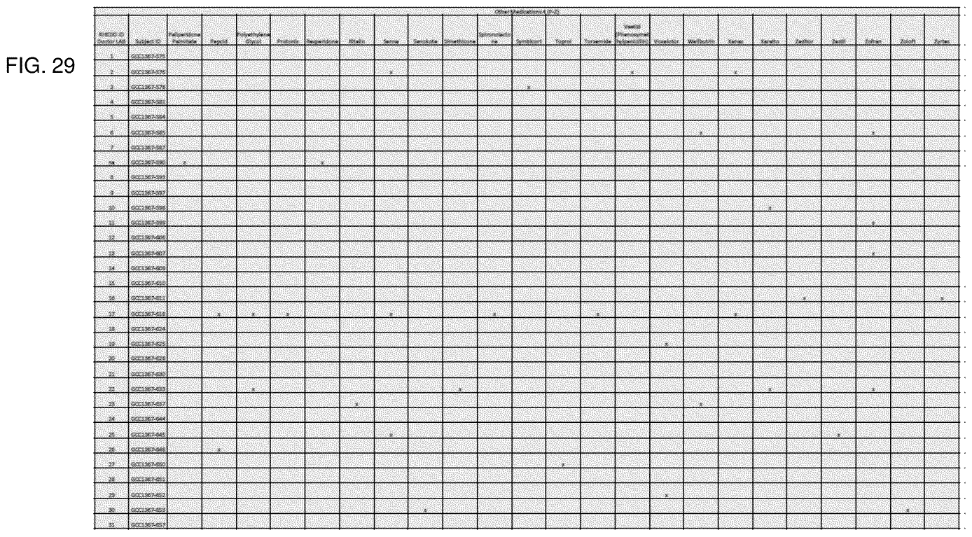

FIG. 24 shows the patient supplement intake; FIG. 25 shows patient analgesic use. See FIG. 26, FIG. 27, FIG. 28, and FIG. 29 for patient medications as indicated.

Table 2, below presents information on example patients/donors 1-6.

TABLE 2
Example Patient Medications

| Donor | Supplements | Analgesic | Other |
|---|---|---|---|
| 1 | Ferrous sulfate | Motrin | NA |
| | Folic acid | | |
| | Vitamin C | | |
| 2 | Folic acid | NA | Colace |
| | Glutamine | | Senna |
| | Vitamin B12 | | Phenoxymethylpenicillin |
| | | | Xanax |
| 3 | Folic acid | Oxycodone | Albuterol |
| | Glutamine | | Symbicort |
| 4 | Folic acid | Toradol | Hydrea (hydroxycarbamide) |
| | Glutamine | | |
| 5 | NA | Motrin | Lisinopril |
| | | Oxycodone | Biktarvy |
| | | | (bictegravir/emtricitabine/ |
| | | | tenofovir alafenamide) |
| 6 | Folic acid | Dilaudid | Deproprovera |
| | | morphine | Hydrea (hydroxycarbamide) |
| | | | JunelFe |
| | | | Wellbutrin |
| | | | Zofran |

### Example 11: Functional Evaluation of Sickle Cell Disease Patients

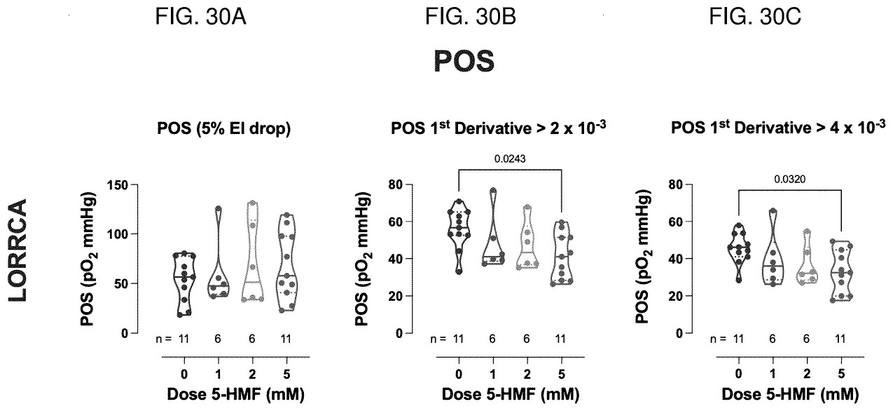

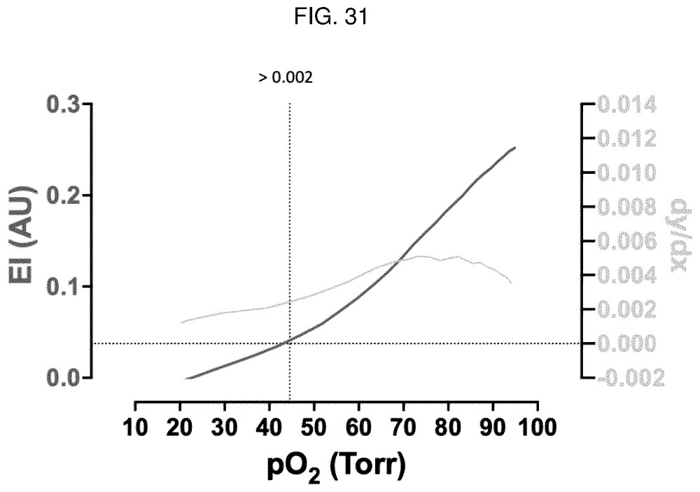

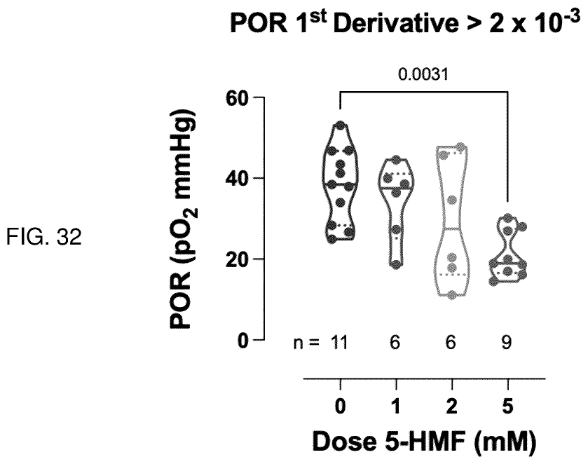

Extensive functional evaluation of these nanoparticles was performed in blood samples collected from SCD patients. The results from the functional studies are given in FIG. 30 (LORRCA determines POS as the pO₂ corresponding to a 5% decrease in EIₘₐₓ. If the EI is not completely stable at high pO₂ (i.e., the Oxyscan baseline is not level), POS is calculated to be artificially high), FIG. 31, and FIG. 32.

### Example 12: Healthy Patient Blood Analysis

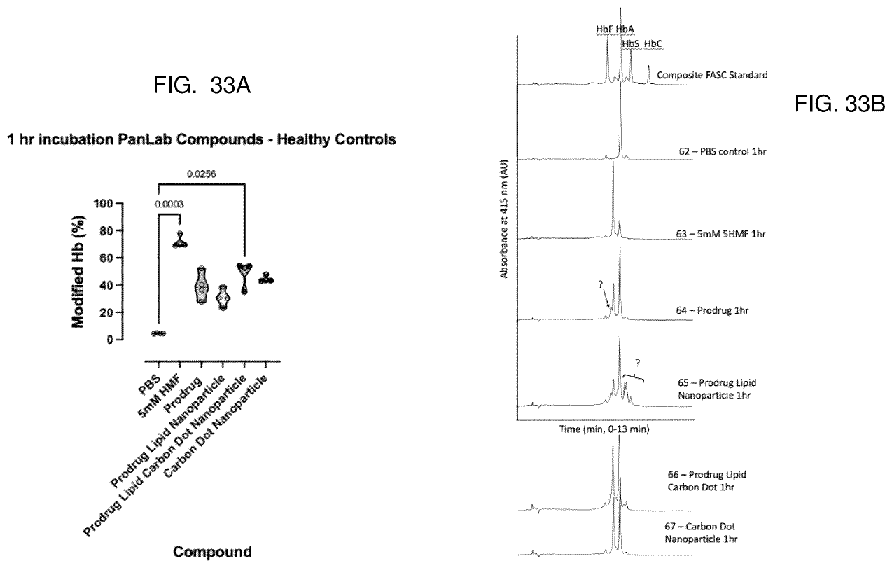

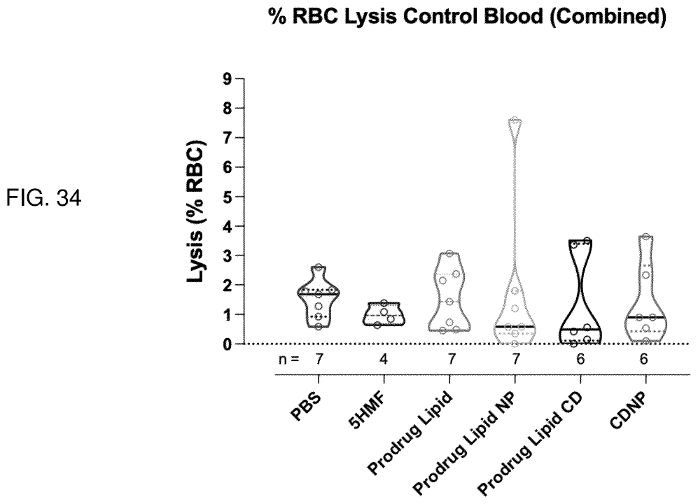

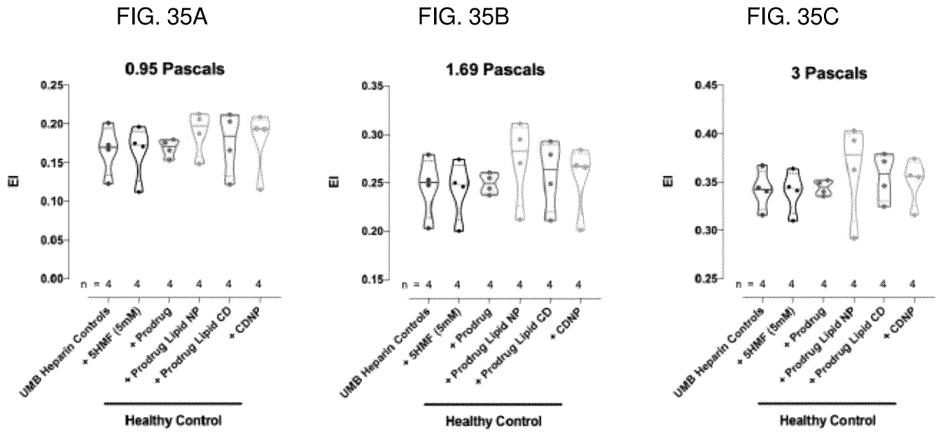

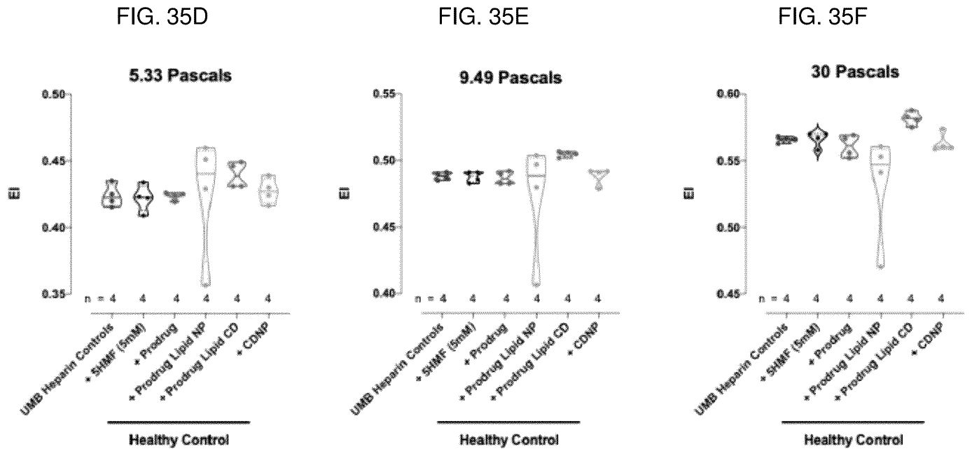

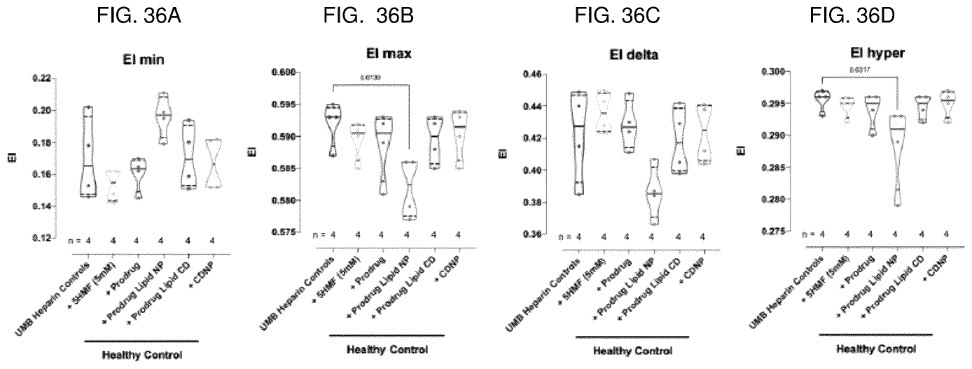

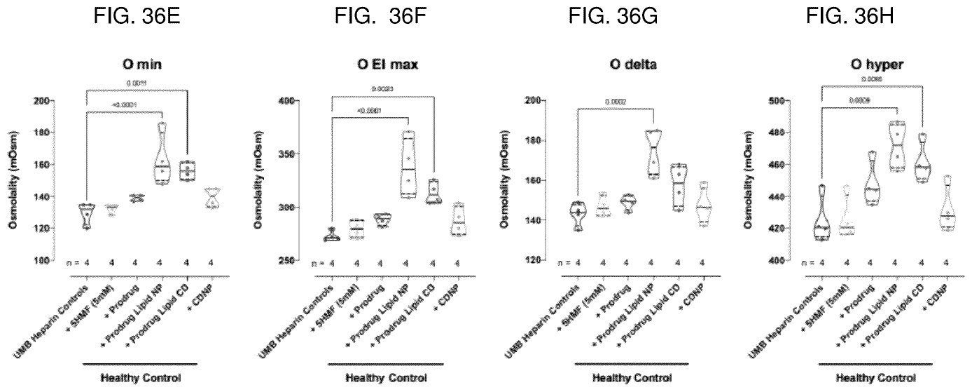

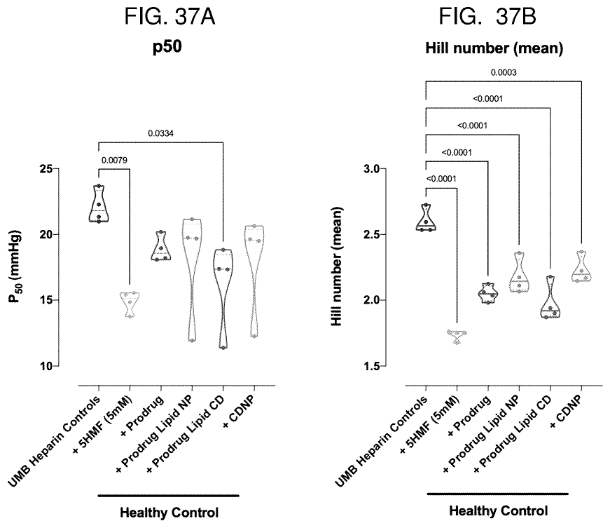

Blood samples (RBCs) from subjects without sickle cell disease were tested with compounds to determine whether the compounds have any impact on RBC physiology, including tests for lysis, LORRCA (deformability and osmotic fragility), and HEMOX (ODC (p50) and cooperativity (Hill number)) after a 1-hour incubation at 37° C. See FIG. 33 for results for HPLC analysis of Hb modification; FIG. 34 for results for % RBC lysis for control blood (combined); FIG. 35 for results as shown; FIG. 36 for results for RBC osmotic fragility; and FIG. 37 for p50 and Hill number results for the oxygen dissociation curve (ODC).

The results show that lysis appears resolved—there was no difference between the PBS control, 5-HMF or the (PanLab) compounds here. There was a small effect in RBC deformability for the prodrug Lipid NP and prodrug Lipid CD, although not in the physiologic range of sheer. Prodrug lipid NP and CD significantly affected RBC osmotic fragility, suggesting that membrane composition/integrity and/or RBCs size or surface-area-to-volume ratio is being affected by these compounds. In the HEMOX studies, the PanLab compounds shifted p50, though not as consistently as HMF. PLA2 should be activating the compounds, so the small shift is expected compared to 5-HMF positive control, as PLA2 levels should be low in healthy controls.

### Example 13: Sickle Cell Patient Blood Analysis

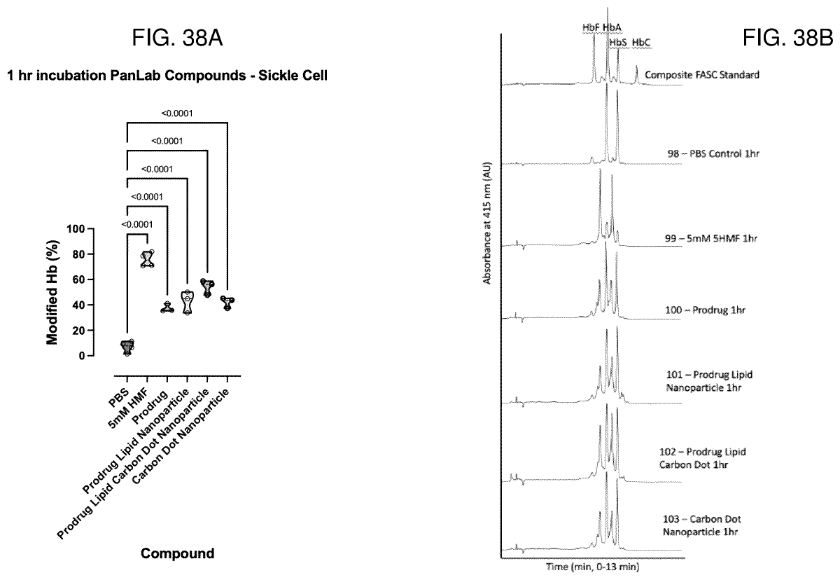

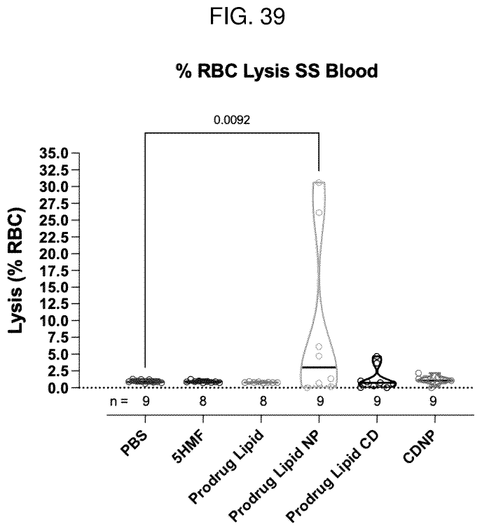

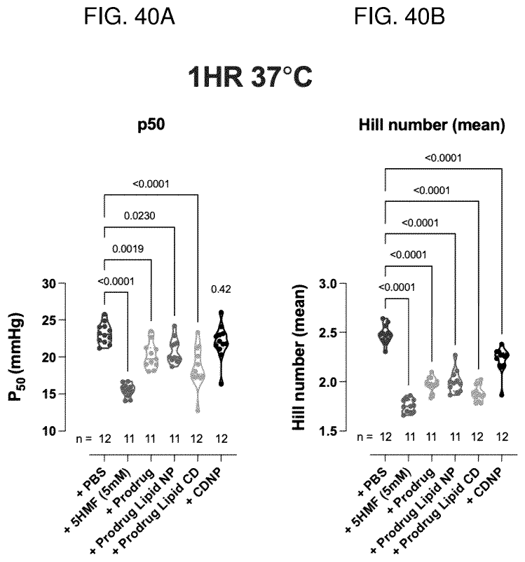

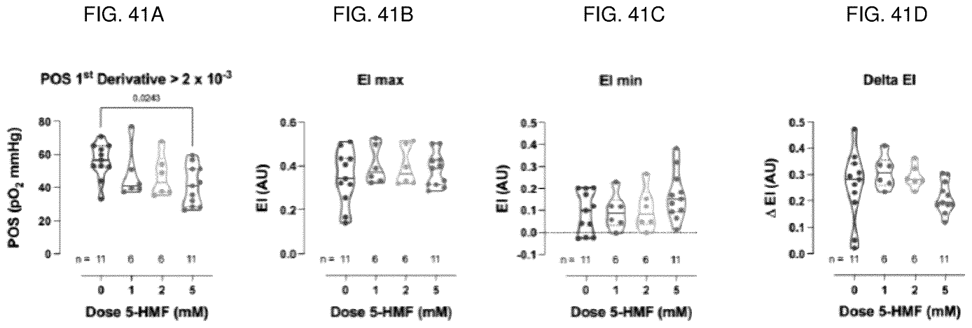

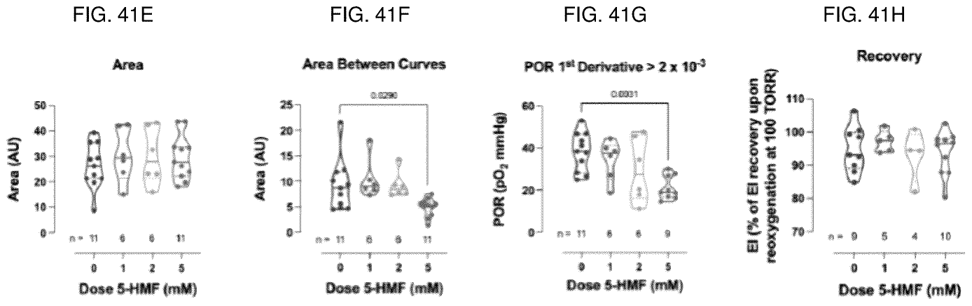

Sickle Cell patient blood samples were tested with PanLab compounds (1-hour incubation at 37° C.). See FIG. 38 for results for Hb modification; FIG. 39 for results for RBC lysis; FIG. 40 for p50 and Hill number results for the oxygen dissociation curve (ODC); and FIG. 41 for results for LORRCA Oxyscan.

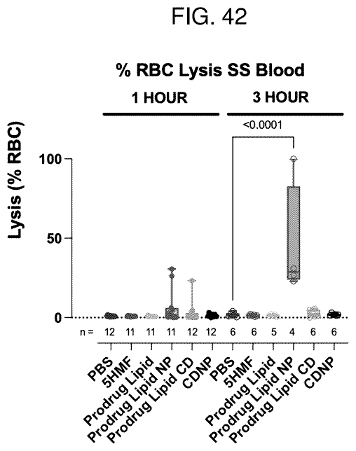

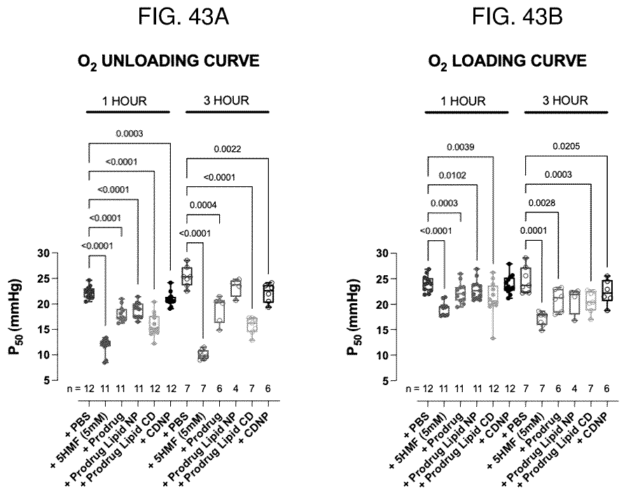

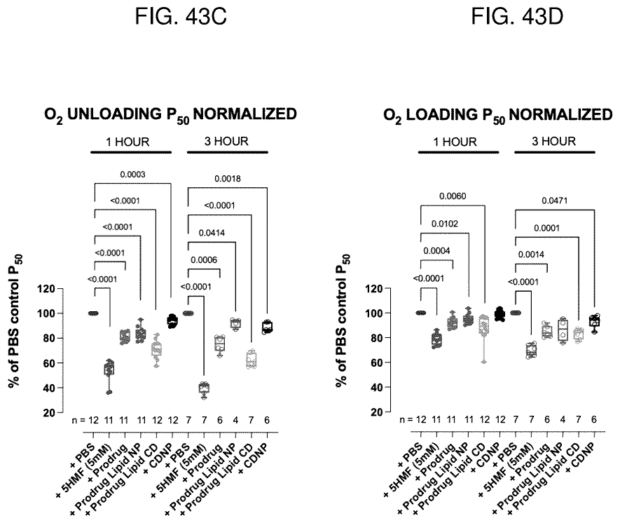

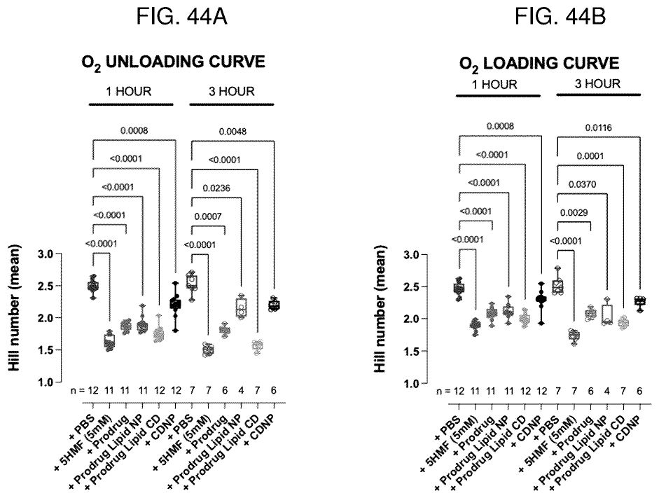

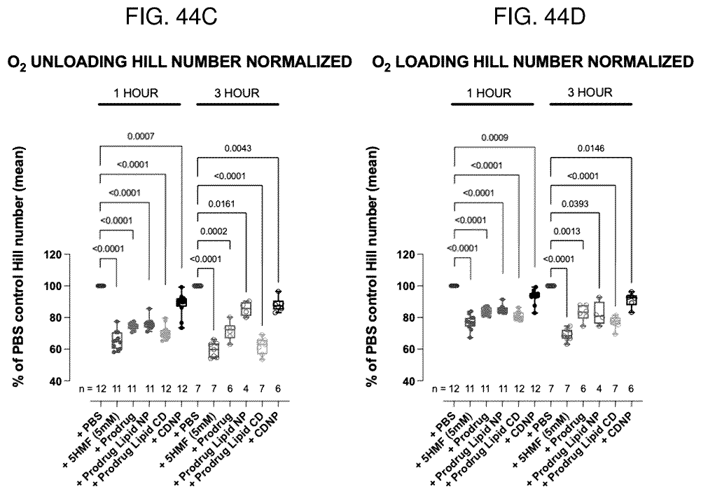

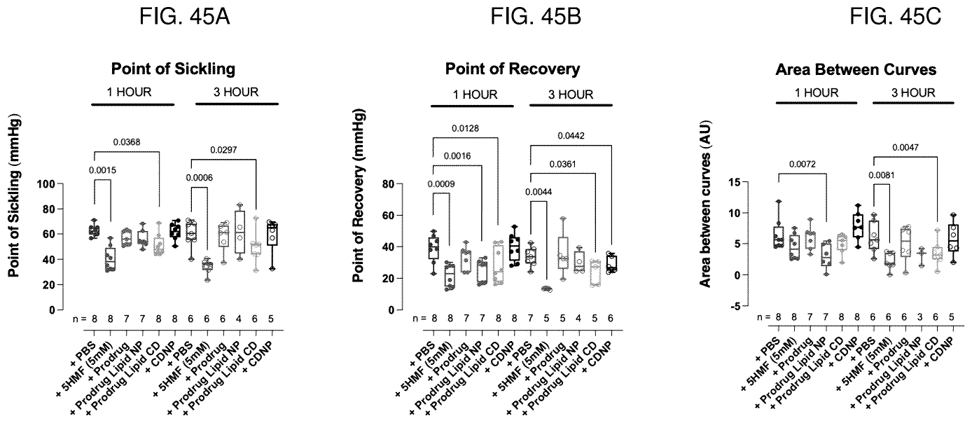

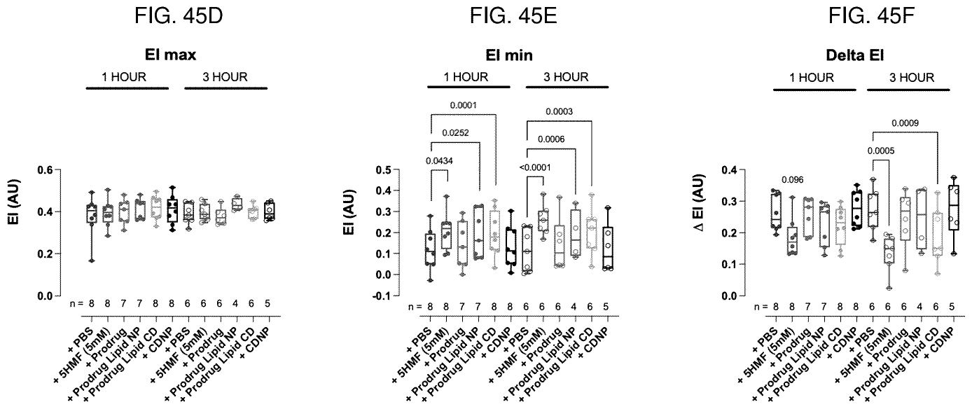

See also FIG. 42 for data on the lysis of sickle cell blood cells, FIG. 43 and FIG. 44 for data on oxygen loading and unloading (all compounds have the equivalent of 5 mM 5-HMF (final)). FIG. 45 presents LORRCA data.

### Example 14: Mouse Pharmacodynamics

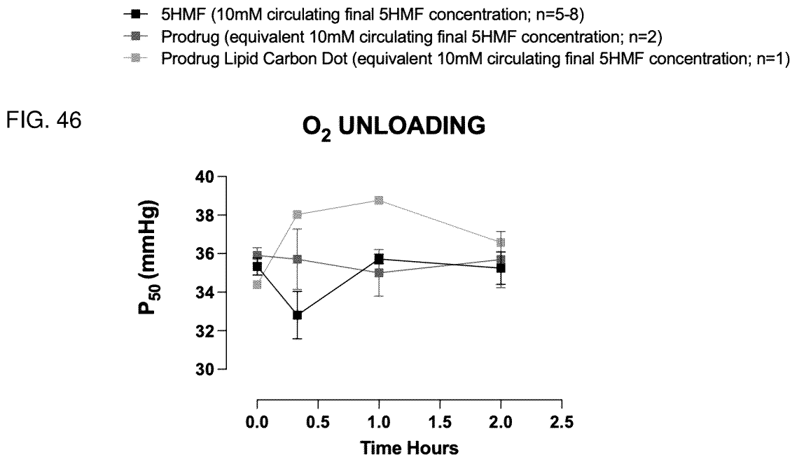

Time started following the full infusion volume 250 μL infusion over 5 minutes through the button. See FIG. 46.

### Example 15: Compound Testing

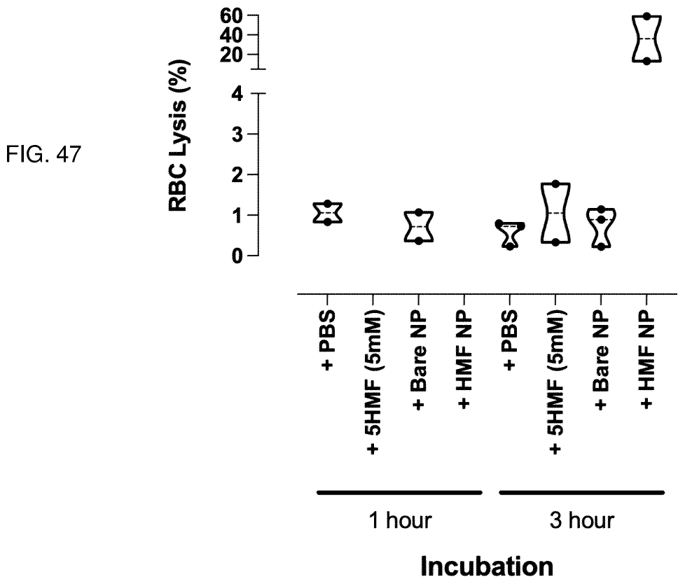

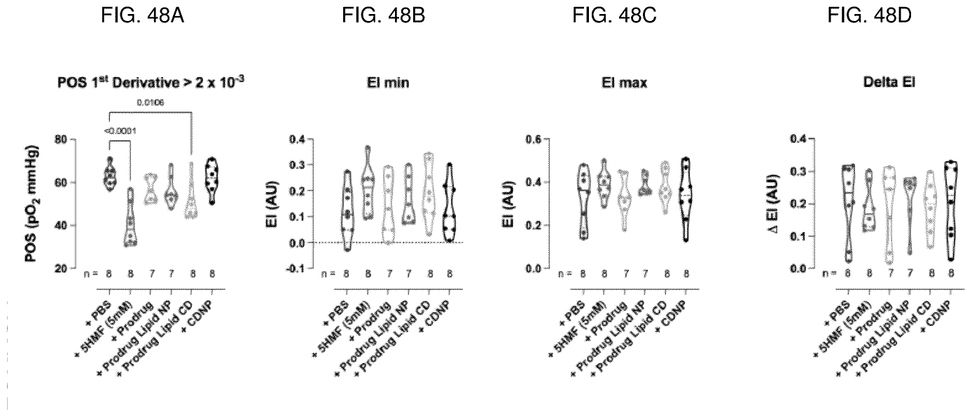

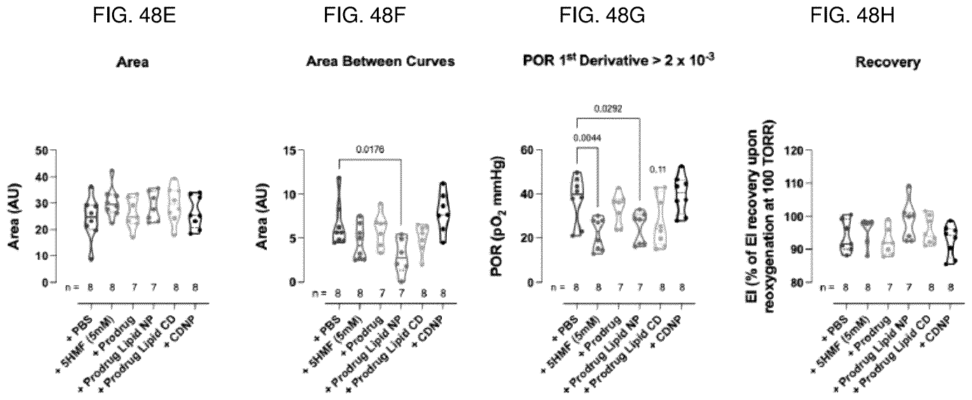

See FIG. 47 for results for RBC lysis; FIG. 48 for results for LORRCA osmotic fragility; and FIG. 48 for results for ODC-HEMOX (p50 and Hill number).

### Example 16: Western Blotting

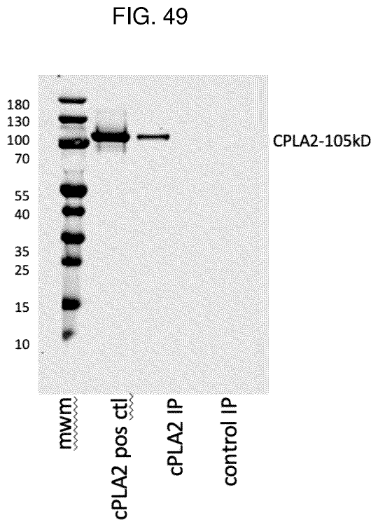

Using magnetic beads, a bead slurry was prepared 1:1 in PBS. To attach antibody to the beads, 30 μL of Sepharose™ bead slurry was added to 2 μL anti-cPLA2 (MBS618221). The antibody was covalently attached. A western blot was performed. See FIG. 49. cPLA2 ran at 105 kD. The blot contained a molecular weight ladder and three lanes. Lane 1=recombinant cPLA2 added to gel 1 μL (0.9 μg/mL). Lane 2=recombinant cPLA2 added to beads 1 μL (0.9 μg/mL) in 200 μL total volume (PBS) containing 30 μL bead slurry. The beads were washed and stripped by boiling in loading buffer. Lane 3=negative control IP—the same as Lane 2 but no recombinant cPLA2 was added.

### Example 17: Imaging Studies

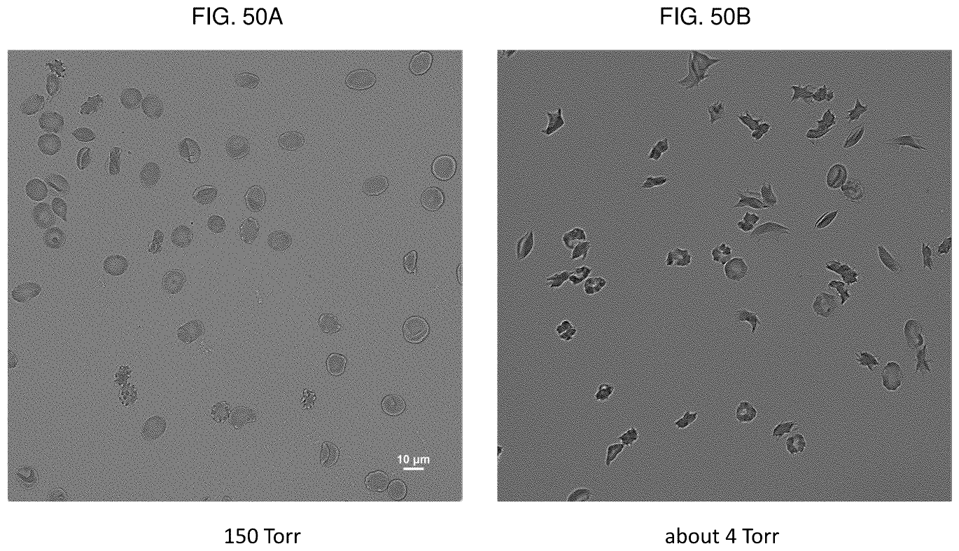

See FIG. 50A and FIG. 50B for imaging studies.

### REFERENCES

All references listed below and throughout the specification are hereby incorporated by reference in their entirety.
1. Abdulmalik et al. 5-hydroxymethyl-2-furfural modifies intracellular sickle haemoglobin and inhibits sickling of red blood cells. Br J Haematol 128, 552-561, 2005.
2. Abraham et al. Toxicology and risk assessment of 5-HMF in food. Mol Nutr Food Res 55, 667-678, 2011.
3. Abraham et al. Vanillin, a potential agent for the treatment of sickle cell anemia. Blood 77, 1334-1341, 1991.
4. Alafeef et al., RNA Extraction-Free Nano-Amplified Colorimetric Test for Point-of-Care Clinical Diagnosis of COVID-19. Nat Protocol. 2021, 14. doi: 10.1038/s41596-021-00546-w.
5. Alafeef et al., Rapid, Ultrasensitive, and Quantitative Detection of SARS-CoV-2 Using Antisense Oligonucleotides Directed Electrochemical Biosensor Chip ACS Nano. 14, 17028-17045, 2020.
6. Alafeef et al., Carbon Dots: from Synthesis to Unraveling the Fluorescence Mechanism. Small 2303937, 2023.
7. Alhashimi et al., Design, Synthesis, and Antisickling Investigation of Nitric Oxide-Releasing Prodrug of 5HMF for the Treatment of Sickle Cell Disease. Biomolecules 1295):696, 2022.
8. Ataga et al. Crizanlizumab for the Prevention of Pain Crises in Sickle Cell Disease. N Engl J Med 376,429-439, 2017.
9. Bennett-Guerrero et al., Evolution of adverse changes in stored RBCs. Proc Natl Acad Sci. 104(43):17063-8, 2007. PubMed PMID: 17940021; PubMed Central PMCID: PMC2040393.
10. Bin et al., Effects of nitroglycerin on erythrocyte rheology and oxygen unloading: novel role of S-nitrosohemoglobin in relieving myocardial ischemia. Circulation. 113(21):2502-8, 2006. PubMed PMID: 16717147.
11. Burkholder et al., Health Evaluation of Experimental Laboratory Mice. Curr Protoc Mouse Biol 2, 145-165, 2012.
12. Cieri-Hutcherson et al., Systematic Review of 1-glutamine for Prevention of Vaso-occlusive Pain Crisis in Patients with Sickle Cell Disease. Pharmacotherapy 39, 1095-1104, 2019.
13. Dalton et al., Nicholson C. Association of bleeding and thrombosis with outcome in extracorporeal life support. Pediatr Crit Care Med. 16(2):167-74, 2015. PubMed PMID: 25647124.
14. Datta et al., Orthogonal self-assembly of an organoplatinum (II) metallacycle and cucurbit[8]uril that delivers curcumin to cancer cells. Proc Natl Acad Sci USA. 115(32), 8087-8092, 2018.
15. Deshpande et al. Rational modification of vanillin derivatives to stereospecifically destabilize sickle hemoglobin polymer formation. Acta Crystallogr D Struct Biol 74, 956-964, 2018.
16. Doctor et al., Hemoglobin conformation couples erythrocyte S-nitrosothiol content to O2 gradients. Proc Natl Acad Sci. 102(16):5709-14, 2005. PubMed PMID: 15824313.
17. Doctor and Stamler, Nitric oxide transport in blood: a third gas in the respiratory cycle. Compr Physiol. (1):541-68, 2011. PubMed PMID: 23737185.
18. Doctor et al., Gaston B. Hemoglobin conformation couples erythrocyte S-nitrosothiol content to O2 gradients. Proc Natl Acad Sci. 102(16):5709-14, 2005. PubMed PMID: 15824313; PubMed Central PMCID: PMC556285.
19. Dufu et al., GBT440 improves red blood cell deformability and reduces viscosity of sickle cell blood under deoxygenated conditions. Clin Hemorheol Microcirc 70, 95-105, 2018.
20. Eaton and Hofrichter, Hemoglobin S gelation and sickle cell disease. Blood 70, 1245-1266, 1987.
21. Fathi et al., Biodegradable Biliverdin Nanoparticles for Efficient Photoacoustic Imaging. ACS Nano. 13(7):7690-7704, 2019.
22. Fathi et al., Computed tomography-guided additive manufacturing of Personalized Absorbable Gastrointestinal Stents for intestinal fistulae and perforations. Biomaterials 228:119542, 2020.
23. Ferlito et al., Hydrogen sulfide increases survival during sepsis: Protective effect of CHOP inhibition. J Immunol. 192(4): 1806-1814, 2014. Doi: 10.4049/jimmunol.1300835
24. Ford et al., Probing single-cell oxygen reserve in sickled erythrocytes via in vivo photoacoustic microscopy. Am J Hematology. 2021; 97(1):E11-E14, 2021. PMID: 34687466
25. Gartia et al., Point-of-service, quantitative analysis of ascorbic acid in aqueous humor for evaluating anterior globe integrity. Sci Rep. 5:16011, 2015.
26. Godfrey et. al., Distribution and metabolism of (5-hydroxymethyl)furfural in male F344 rats and B6C3F1 mice after oral administration. J Toxicol Environ Health A 57, 199-210, 1999.
27. Guo et al., Early Thalamocortical Reperfusion Leads to Neurologic Recovery in a Rodent Cardiac Arrest Model. Neurocritical Care 2022. doi: 10.1007/s12028-021-01432-9
28. Hamideh et al., Biodegradable MRI Visible Drug Eluting Stent Reinforced by Metal Organic Frameworks. Adv Healthc Mater. July; 9(14):e2000136, 2020.
29. Hannemann et al., Effects of 5-hydroxymethyl-2-furfural on the volume and membrane permeability of red blood cells from patients with sickle cell disease. J Physiol 592, 4039-4049, 2014.
30. Hansen et al., 5-(Hydroxymethyl)furfural restores low-oxygen rheology of sickle trait blood in vitro. Br J Haematol 188, 985-993, 2020.
31. Horiuchi et. al., Morphologic studies of sickle erythrocytes by image analysis. J Lab Clin Med 115, 613-620, 1990.
32. Howard et al., Voxelotor in adolescents and adults with sickle cell disease (HOPE): long-term follow-up results of an international, randomised, double-blind, placebo-controlled, phase 3 trial. Lancet Haematol 8, e323-e333, 2021.
33. Janocha et al., Nitric oxide during altitude acclimatization. N Engl J Med 365(20):1942-4, 2011. PubMed PMID: 22087700.
34. Janzowski et al., 5-Hydroxymethylfurfural: assessment of mutagenicity, DNA-damaging potential and reactivity towards cellular glutathione. Food Chem Toxicol 38, 801-809, 2000.
35. Kato et.al., Phase 1 Clinical Trial of Aes-103 in Adults with Sickle Cell Anemia. Blood 122, 1009, 2013.
36. Kim et al., Sensitive Biological Detection with a Soluble and Stable Polymeric Paramagnetic Nanocluster. J. Am. Chem. Soc. 134(25):10377-80, 2012.
37. Li et al., The Structural Characteristics and Expression of Neuropeptides in the Esophagus of Patients with Congenital Esophageal Atresia and Tracheoesophageal Fistula. Journal of Pediatric Surgery. 2007; 42(8): 1433-38, 2007. ncbi.nlm.nih.gov/pubmed/17706510
38. Mai et al., Body temperature and mouse scoring systems as surrogate markers of death in cecal ligation and puncture sepsis. Intensive Care Med Exp 6:20, 2018.
39. Manci et al., Pathology of Berkeley sickle cell mice: similarities and differences with human sickle cell disease. Blood 107, 1651-1658, 2006.
40. Marozkina et al., Transnitrosation signals oxyhemoglobin desaturation. Circ Res. 103(5):441-3, 2008. PubMed PMID: 18757830; PubMed Central PMCID: PMC2738832.
41. McMahon and Doctor, Extrapulmonary effects of inhaled nitric oxide: role of reversible S-nitrosylation of erythrocytic hemoglobin. Proc Am Thorac Soc. 3(2):153-60, 2006. PubMed PMID: 16565424.
42. Metcalf et al., Discovery of GBT440, R-State Stabilizer of Sickle Hb. ACS Med Chem Lett 8, 321-326, 2017.
43. Misra et al., Trimodal Therapy: Combining Hyperthermia with Repurposed Bexarotene and Ultrasound for Treating Liver Cancer. ACS Nano. 9(11):10695-718, 2015.
44. Misra et al., Macromolecularly “Caged” Carbon Nanoparticles for Intracellular Trafficking via Switchable Photoluminescence. J Am Chem Soc. 139(5):1746-1749, 2017.
45. Misra et al., Hyperspectral Imaging Offers Visual and Quantitative Evidence of Drug Release from Zwitterionic-Phospholipid-Nanocarbon When Concurrently Tracked in 3D Intracellular Space. Advanced Functional Materials 26 (44), 8031-8041, 2016.
46. Misra et al., Systemic administration of dendrimer N-acetyl cysteine improves outcomes and survival following cardiac arrest. Bioengineering & Translational Medicine 7(1), 2021. aiche.onlinelibrary.wiley.com/doi/10.1002/btm2.10259.
47. Modi et al., Intranasal post-cardiac arrest treatment with Orexin-A facilitates arousal from coma and ameliorates neuroinflammation. PLoS One. 12(9), 2017. ncbi.nlm.nih.gov/pubmed/28957432.
48. Moitra P, Alafeef M, Dighe K, Frieman M B, Pan D*. Selective Naked-Eye Detection of SARS-CoV-2 Mediated by N Gene Targeted Antisense Oligonucleotide Capped Plasmonic Nanoparticles. ACS Nano. 2020 14, 6, 7617-7627.
49. Nguyen et al., Ultrafast nanometric imaging of energy flow within and between single carbon dots. Proc Natl Acad Sci USA. 118(11):e2023083118, 2021.
50. Oder et al., New developments in anti-sickling agents: can drugs directly prevent the polymerization of sickle haemoglobin in vivo? Br J Haematol 175:24-30, 2016.
51. Oksenberg et al., GBT440 increases haemoglobin oxygen affinity, reduces sickling and prolongs RBC half-life in a murine model of sickle cell disease. Br J Haematol 175:141-153, 2016.
52. Ostadhossein et al., Dual purpose hafnium oxide nanoparticles offer imaging Streptococcus mutans dental biofilm and fight it In vivo via a drug free approach. Biomaterials. 181:252-267, 2018.
53. Ostadhossein et al., Multi-“Color” Delineation of Bone Microdamages Using Ligand-Directed Sub-5 nm Hafnia Nanodots and Photon Counting CT Imaging. Advanced Functional Materials 30, 2020. 1904936.
54. Palmer et al., S-nitrosothiols signal hypoxia-mimetic vascular pathology. J Clin Invest. 117(9):2592-601, 2007. PubMed PMID: 17786245.
55. Pan, 3D-Printed Multidrug-Eluting Stent from Graphene-Nanoplatelet-Doped Biodegradable Polymer Composite. Adv Healthc Mater. 2017 June; 6(11), 2017.
56. Pan et al., Ligand-directed Nanobialys as Theranostic Agent for Drug Delivery and Manganese based Magnetic Resonance Imaging (MRI) of Vascular Targets. J. Am. Chem. Soc. 130 (29): 9186-9187, 2008.
57. Pan et al., Sensitive and efficient detection of thrombus with fibrin-specific manganese nanocolloids. Chem. Commun. 3234-3236, 2009.
58. Pan et al., Folic acid-conjugated nanostructured materials designed for cancer cell targeting. Chem. Commun. 2400-2401, 2003.
59. Piel et al., Sickle Cell Disease. N Engl J Med 376, 1561-1573, 2017.
60. Pineda et al., Effect of implementation of a paediatric neurocritical care programme on outcomes after severe traumatic brain injury: a retrospective cohort study. Lancet Neurol. 12(1):45-52, 2013. PubMed PMID: 23200264.
61. Poillon and Kim, 2,3-Diphosphoglycerate and intracellular pH as interdependent determinants of the physiologic solubility of deoxyhemoglobin S. Blood 76, 1028-1036, 1990.
62. Pollack et al., Relationship between the functional status scale and the pediatric overall performance category and pediatric cerebral performance category scales. JAMA Pediatr. 168(7):671-676, 2014. PubMed PMID: 24862461.
63. Pollack et al., Simultaneous Prediction of New Morbidity, Mortality, and Survival Without New Morbidity From Pediatric Intensive Care: A New Paradigm for Outcomes Assessment. Crit Care Med. 43(8):1699-709, 2015. PubMed PMID: 25985385.
64. Rab et al., Characterization of Sickling During Controlled Automated Deoxygenation with Oxygen Gradient Ektacytometry. J Visualized Experiments: JoVE (2019).
65. Rabiei et al., Nanomaterial and advanced technologies in transdermal drug delivery. J Drug Target 28, 356-367, 2020.
66. Rogers et al. Effect of plasma processing and storage on microparticle abundance, nitric oxide scavenging, and vasoactivity. Transfusion. 59(S2):1568-1577, 2019. PMID: 30980740
67. Rogers et al., Quantifying Dynamic Range in Red Blood Cell Energetics: Evidence of Progressive Energy Failure During Storage. Transfusion. 61(5):1586-1599, 2021. PMID: 33830505.
68. Rogers et al., Hypoxia limits antioxidant capacity in red blood cells by altering glycolytic pathway dominance. FASEB J. 23(9):3159-70, 2009. PubMed PMID: 19417084; PubMed Central PMCID: PMC2735367.
69. Rogers et al., Sickle hemoglobin disturbs normal coupling between erythrocyte O2 content, glycolysis and antioxidant capacity. Blood. 121(9):1651-62, 2003. PMID: 23297128.
70. Ryan and Townes, Knockout-transgenic mouse model of sickle cell disease. Science 278, 873-876, 1997.
71. Safo and Kato, Strategies to alter the O2 affinity of sickle Hb. Hematol Oncol Clin North Am 28, 217-231, 2014.
72. Safo et al., Structural basis for the potent antisickling effect of a novel class of five-membered heterocyclic aldehydic compounds. J Med Chem 47, 4665-4676, 2004.
73. Schwartz-Duval et al., Generation of Photothermal Gold Nanoparticles through a Vectorized Application of Ionic Gold. Nature Communications. 11: 4530, 2020.
74. Senarathna et al., A Miniature Multi-contrast Microscope for Functional Imaging in Freely Behaving Animals. Nature Communications. 10, Article number: 99, 2019.
75. Shrum et al., A robust scoring system to evaluate sepsis severity in an animal model. BMC Res Notes 7, 233, 2014.
76. Singh et al., Low-cost solution for rodent home-cage behaviour monitoring. PLoS One 14, e0220751, 2019)
77. Spinella and Doctor, Role of transfused red blood cells for shock and coagulopathy within remote damage control resuscitation. Shock 41:1:30-34, 2014. PubMed PMID: 24296434.
78. Srivastava et al., Influence of Electron Acceptor and Electron Donor on the Photophysical Properties of Carbon Dots: A Comparative Investigation at the Bulk-State and Single-Particle Level. Advanced Functional Materials. 29(37):1902466, 2019.
79. Srivastava et al., Enzyme-catalysed biodegradation of carbon dots follows sequential oxidation in a time dependent manner. Nanoscale 11, 8226-8236, 2019.
80. Vasiliou et al., Role of aldehyde dehydrogenases in endogenous and xenobiotic metabolism. Chem Biol Interact 129, 1-19, 2000.
81. Vichinsky et al., A Phase 3 Randomized Trial of Voxelotor in Sickle Cell Disease. N Engl J Med 381, 509-519, 2019.
82. Wang et al., Changes in Serum Amylase Isoenzymes in Congenital Biliary Dilatation. Chinese Journal of Pediatric Surgery. 4: 11-13, 2000.
83. Wang et al., Analysis on the Postoperative Short-term Effect and Some Factors Affecting the Prognosis in 57 Cases with Biliary Atresia. J Clinical Pediatrics. 12: 716-718, 2002.
84. Wang et al., The effect of therapeutic hypothermia on brain homeostasis in a cardiac arrest rat model. Journal of Cerebral Blood Flow & Metabolism 39(10):1961-1973, 2019. https://www.ncbi.nlm.nih.gov/pubmed/29739265
85. Wei et al., Acute-stage MRI cerebral oxygen consumption biomarkers predict 24-hour neurological outcome in a rat cardiac arrest model. NMR in Biomedicine 2020. onlinelibrary.wiley.com/doi/abs/10.1002/nbm.4377
86. Wright et al., Protection of Microvascular Liver Perfusion with 5-Hydroxymethylfurfural in Sickle Cell Disease Mice Following Hypoxia-Induced Vasoocclusion. Blood 126, 3382, 2015.
87. Wu et al., A green synthesis of carbon nanoparticles from honey and their use in real-time photoacoustic imaging. Nano Research 6(5): 312-325, 2013.
88. Xiao et al., Potential Diagnostic Value of Pancreatic Isoamylases for Pancreaticobiliary Maljunction with Mild Biliary Dilatation in Patients and A Porcine Model. Journal of Pediatric Surgery. 39(10): 1490-1494, 2004. http://www.ncbi.nlm.nih.gov/pubmed/15486892
89. Xu et al., Design, Synthesis, and Biological Evaluation of Ester and Ether Derivatives of Antisickling Agent 5-HMF for the Treatment of Sickle Cell Disease. Mol Pharm 14, 3499-3511, 2017.
90. Zaugg et.al., Schiff base adducts of hemoglobin inhibit sickling. J Biol Chem 252, 8542-8548, 1977.
91. Zhang et al., Elevated sphingosine-1-phosphate promotes sickling and sickle cell disease progression. J Clin Invest 124, 2750-2761 (2014).
92. U.S. Pat. No. 8,354,093.
93. U.S. Pat. No. 9,446,150.
94. U.S. Pat. No. 9,468,607.
95. U.S. Pat. No. 9,498,439.
96. U.S. Pat. No. 9,486,508.
97. U.S. Pat. No. 9,655,952.
98. U.S. Pat. No. 10,400,262.
99. U.S. Pat. No. 10,201,500.

1. A nanoparticle, comprising:
(a) a carbon core comprising a carbon dot and complexed 5-HMF; and
(b) a 5-HMF-phospholipid shell comprising a lipid complexed to 5-HMF,
wherein the lipid complex is cleavable by PLA2.

2. The nanoparticle of claim 1 which is about 0.1 nm to about 200 nm in diameter.

3. The nanoparticle of claim 1 which is about 1 nm to about 100 nm in diameter.

4. The nanoparticle of claim 1 which is about 50 nm to about 100 nm in diameter.

5. The nanoparticle of claim 1, wherein the phospholipid is an oxidized lipid.

6. The nanoparticle of claim 1, wherein the phospholipid is 1-lysophosphatidylcholine.

7. A nanoparticle pharmaceutical composition comprising a pharmaceutically acceptable vehicle and the nanoparticle of claim 1.

8. The nanoparticle pharmaceutical composition of claim 7, wherein the composition is formulated for transdermal administration.

9. A method of treating sickle cell disease in a subject in need thereof, comprising administering to the subject the nanoparticle of claim 1.

10. A method of treating sickle cell disease in a subject in need thereof, comprising administering to the subject the nanoparticle pharmaceutical composition of claim 7.
